(19) United States
(12) Patent Application Publication
(10) Pub. No.: US 2021/0290537 A1
(43) Pub. Date: Sep. 23, 2021

(54) LIPOSOMAL ELINAFIDE FORMULATIONS AND USES THEREOF

(71) Applicant: MALLINCKRODT LLC, Hazelwood, MO (US)

(72) Inventors: William McGHEE, Fenton, MO (US); Rick FITCH, O'Fallon, MO (US)

(21) Appl. No.: 16/467,458

(22) PCT Filed: Dec. 8, 2017
(86) PCT No.: PCT/US2017/065229

§ 371 Date: Jun. 6, 2019

Related U.S. Application Data
(60) Provisional application No. 62/431,524, filed on Dec. 8, 2016.

(51) Int. Cl. A61K 9/127; A61K 9/00; A61K 31/473; A61P 35/00

(52) U.S. Cl. CPC A61K 9/1271; A61P 35/00; A61K 31/473; A61K 9/0019

(57) ABSTRACT

The present invention provides compositions for the treatment of cancer. The compositions include liposomes containing a phosphatidylcholine lipid, a sterol, a PEG-lipid, and elinafide, wherein less than 20% of the elinafide is released in vitro from the liposome within 40 hours. The present invention also provides liposomal compositions for the treatment of cancer comprising administering to a patient in need thereof a liposome, wherein the liposome comprises: a phosphatidylcholine lipid; a sterol; a PEG-lipid; and elinafide, wherein the administration of the liposomal composition has a reduced occurrence of side effects, such as for example, muscle myopathy in the patient.

### CLAIM OF PRIORITY

This application claims priority to U.S. Provisional Application No. 62/431,524 filed on Dec. 8, 2016. The entire content of the above-referenced application is incorporated by reference herein.

### BACKGROUND OF THE INVENTION

Naphthalimides and azonifides, classes of compounds that bind to DNA by intercalation, have been shown to possess anti-tumor activity. Both naphthalimides and azonifides are also known to interact with topoisomerases I and/or II, and such agents are known to have anti-tumor activity. Naphthalimides and azonifides are structurally related and shown below.

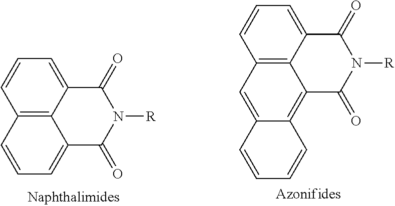

Despite decades of extensive research on the utility of naphthalimides and azonifides as anti-cancer agents, none is currently approved for therapeutic use, e.g., cancer therapy. There has been a number of clinical trials, but due to dose-limiting toxicity, none has ever acceded to market. For instance, a phase I study of elinafide (LU79553) demonstrated cumulative dose-limiting neuro-muscular toxicity and myelosuppression at doses at or below efficacy. Similarly, phase I studies of mitonafide, UNBS-5162, and DMP-840 showed dose limiting toxicity, including myelosuppression, irreversible CNS toxicity, QTc prolongation, neutropenia, thrombocytopenia and/or stromatitis. Recently, amonafide (AS1413) failed to enter phase III clinical trials due to high variability and dose-limiting toxicity, including myelosuppression and neutropenia.

The structures of these mono- and di-naphthalimides and azonifides are shown below.

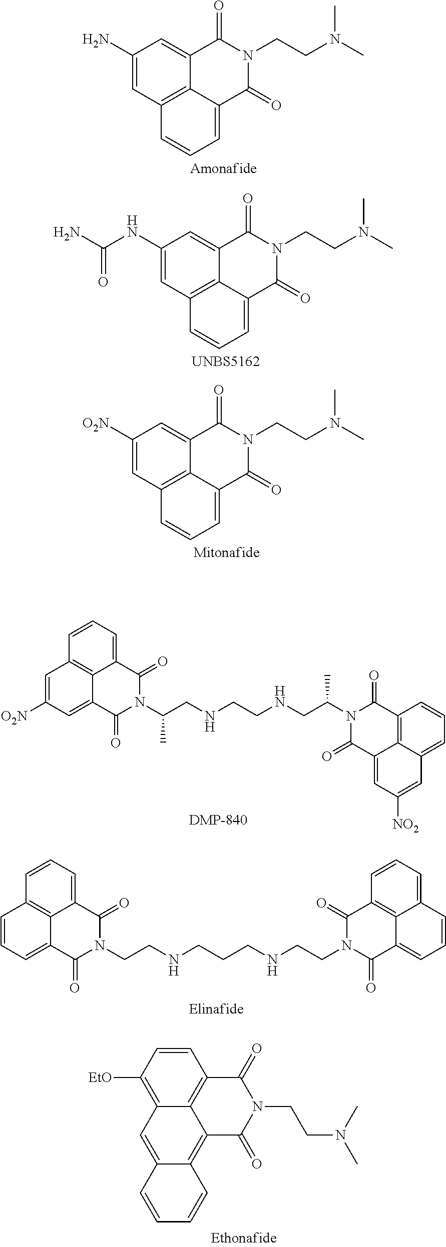

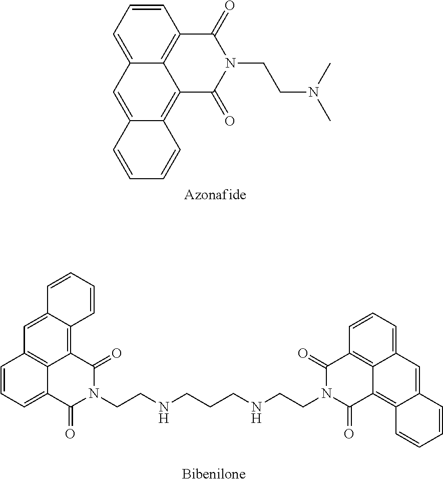 [SMILES: CN(C)CCN1C(=O)c2cccc3cc4ccccc4c(c23)C1=O ; O=C1c2cccc3cc4ccccc4c(c23)C(=O)N1CCNCCCNCCN1C(=O)c2cccc3cc4ccccc4c(c23)C1=O]

Accordingly, there exists a need in the art for a naphthalimide or azonifide derivative as an anti-cancer agent having reduced dose-limiting toxicity and/or improved efficacy. The present invention provides a liposomal elinafide composition that addresses this and other such needs.

### BRIEF SUMMARY OF THE INVENTION

In a first aspect, the present invention provides a liposome containing a phosphatidylcholine lipid, a sterol, a poly(ethylene glycol)-phospholipid conjugate (PEG-lipid), and elinafide. In a further aspect, less than about 20% of elinafide is released in vitro from the liposome within about 40 hours.

In a second aspect, the present invention provides a liposomal elinafide composition for the treatment of cancer. The composition includes a liposome containing a phosphatidylcholine lipid, a sterol, PEG-lipid, and elinafide, and a pharmaceutically acceptable excipient.

In a third aspect, the present invention provides a method for preparing liposomal elinafide. The method includes: a) forming a first liposome having a lipid bilayer including a phosphatidylcholine lipid and a sterol, wherein the lipid bilayer encapsulates an interior compartment or space containing an aqueous solution; b) loading the first liposome with elinafide, or a pharmaceutically acceptable salt thereof, to form a loaded liposome; and, optionally c) forming a mixture containing the loaded liposome and a PEG-lipid under conditions sufficient to allow insertion of the PEG-lipid into the lipid bilayer.

In a fourth aspect, the present invention provides a method for treating cancer comprising administering to a patient in need thereof the liposomal elinafide composition of the invention.

In a fifth aspect, the method for treating cancer has reduced side effects commonly associated with free elinafide, including muscle myopathy, myelosuppression, neuromuscular toxicity, and QTc prolongation.

### BRIEF DESCRIPTION OF THE DRAWINGS

FIG. 1 shows cryo-TEMs of various elinafide liposomes. FIG. 1A represents a cryo-TEM of example 1a shown at 52,000× magnification (scale bar: 200 nm). FIG. 1B represents a cryo-TEM of example 1b shown at 52,000× magnification (scale bar: 200 nm). FIG. 1C represents a cryo-TEM of example 1c shown at 52,000× magnification (scale bar: 200 nm). FIG. 1D represents a cryo-TEM of example 1d shown at 52,000× magnification (scale bar: 200 nm).

FIG. 1E represents a cryo-TEM of example 1e shown at 52,000× magnification (scale bar: 200 nm). FIG. 1F represents a cryo-TEM of example 1f shown at 52,000× magnification (scale bar: 200 nm).

FIG. 2A shows the in vitro release of elinafide from the liposome in fetal bovine serum at 37° C. over time for examples 1a, 1b, and 1c. FIG. 2B shows the in vitro release of elinafide from the liposome in fetal bovine serum at 37° C. over time for examples 1d, 1e, and 1f.

FIG. 3A shows the mean tumor volume of A549 human NSCLC xenografts in athymic nude mice after a single intravenous administration of saline, free elinafide, or liposomal elinafide. FIG. 3B shows the mean tumor volume of HT29 human colorectal xenografts in athymic nude mice after a single intravenous administration of saline, free elinafide, or liposomal elinafide. FIG. 3C shows the mean tumor volume of BxPC3 human pancreatic xenografts in athymic nude mice after a single intravenous administration of saline, free elinafide, or liposomal elinafide. All data are represented as mean f standard error of 5 to 10 mice.

FIG. 4A shows the mean tumor volume of HT29 human colorectal xenografts in athymic nude mice after a single intravenous administration of liposomal elinafide, Abraxane, or DOXIL. FIG. 4B shows the median survival of athymic nude mice bearing HT29 human colorectal xenografts after a single intravenous administration of liposomal elinafide, Abraxane, or DOXIL. All data are represented as mean f standard error of 5 to 10 mice.

FIG. 5A shows the plasma concentration of elinafide over time in athymic nude mice bearing HT29 human colorectal xenograft tumors following a single intravenous administration of liposomal elinafide or free elinafide. FIG. 5B shows the tumor concentration of elinafide over time in HT29 human colorectal xenograft tumors harvested from athymic nude mice following a single intravenous administration of liposomal elinafide or free elinafide. All data are represented as mean f standard error of 3 mice, with the exception of the 120 hr time point which contained only 2 mice in the liposomal elinafide group.

FIG. 6 shows histological sections of the quadriceps femoris of Sprague-Dawley male rats after a single intravenous administration of 50 mg/kg liposomal elinafide or 25 mg/kg elinafide. The top images are cross sections of muscle from the right leg and the bottom images are longitudinal sections of the left leg. Magnification is at 20×.

FIG. 7A shows cryo-TEM of liposomal amonafide (sulfate) at 52,000× magnification (scale bar: 200 nm). FIG. 7B shows cryo-TEM of liposomal amonafide (citrate) at 52,000× magnification (scale bar: 200 nm). FIG. 7C shows cryo-TEM of liposomal UNBS-5162 (sulfate) at 52,000× magnification (scale bar: 200 nm).

FIG. 8A shows the percent change in mean tumor volume of HT29 human colorectal xenografts in athymic nude mice after a single intravenous administration of amonafide, liposomal amonafide (citrate) or liposomal amonafide (sulfate). All data are represented as mean f standard error of 3 to 5 mice.

FIG. 9A shows the percent change in mean tumor volume of HT29 human colorectal xenografts in athymic nude mice after a single intravenous administration of liposomal amonafide (citrate), liposomal amonafide (sulfate), azonafide, liposomal azonafide (citrate), and liposomal azonafide (sulfate). FIG. 9B shows the percent change in mean tumor volume of HT29 human colorectal xenografts in athymic nude mice after a single intravenous administration of amonafide, liposomal amonafide (citrate), liposomal amonafide (sulfate), azonafide, liposomal azonafide (citrate), and liposomal azonafide (sulfate). All data are represented as mean f standard error of 3 to 5 mice.

FIG. 10A shows the percent change in mean tumor volume of HT29 human colorectal xenografts in athymic nude mice after a single intravenous administration of ethonafide, liposomal ethonafide (citrate), and liposomal ethonafide (sulfate). FIG. 10B shows the percent change in mean tumor volume of HT29 human colorectal xenografts in athymic nude mice after a single intravenous administration of ethonafide, liposomal ethonafide (citrate), liposomal ethonafide (sulfate), UNBS-5126, liposomal UNBS-5126 (citrate), and liposomal UNBS-5126 (sulfate). All data are represented as mean f standard error of 3 to 5 mice.

### DETAILED DESCRIPTION OF THE INVENTION

### I. General

The present invention provides novel elinafide liposomes and compositions thereof. The liposomal elinafide compositions herein demonstrates several advantages including improved anti-tumor efficacy, increased plasma circulation time and tumor accumulation, and reduced dose-limiting toxicity. Further, the liposomal elinafide composition of the present invention is useful for the treatment of cancer.

### II. Definitions

As used herein, the term “liposome” encompasses any compartment enclosed by a lipid bilayer. The term liposome includes unilamellar vesicles which are comprised of a single lipid bilayer and generally have a diameter in the range of about 20 to about 400 nm. Liposomes can also be multilamellar, which generally have a diameter in the range of 1 to 10 μm. In some embodiments, liposomes can include multilamellar vesicles (MLVs; from about 1 μm to about 10 μm in size), large unilamellar vesicles (LUVs; from a few hundred nanometers to about 10 μm in size), and small unilamellar vesicles (SUVs; from about 20 nm to about 200 nm in size).

As used herein, the term “phosphatidylcholine lipid” refers to a diacylglyceride phospholipid having a choline headgroup (i.e., a 1,2-diacyl-sn-glycero-3-phosphocholine). The acyl groups in a phosphatidylcholine lipid are generally derived from fatty acids having from 6 to 24 carbon atoms. Phosphatidylcholine lipids can include synthetic and naturally-derived 1,2-diacyl-sn-glycero-3-phosphocholines.

As used herein, the term “sterol” refers to a steroid containing at least one hydroxyl group. A steroid is characterized by the presence of a fused, tetracyclic gonane ring system. Sterols include, but are not limited to, cholesterol (i.e., 2,15-dimethyl-14-(1,5-dimethylhexyl)tetracyclo[8.7.0.0^{2,7}.0^{11,15}]heptacos-7-en-5-ol; Chemical Abstracts Services Registry No. 57-88-5).

As used herein, the term “PEG-lipid” refers to a poly(ethylene glycol) polymer covalently bound to a hydrophobic or amphipilic lipid moiety. The lipid moiety can include fats, waxes, steroids, fat-soluble vitamins, monoglycerides, diglycerides, phospholipids, and sphingolipids. Preferred PEG-lipids include diacyl-phosphatidylethanolamine-N-[methoxy(polyethene glycol)]s and N-acyl-sphingosine-1-{succinyl[methoxy(polyethylene glycol)]}s. The molecular weight of the PEG in the PEG-lipid is generally from about 500 to about 5000 Daltons (Da; g/mol). The PEG in the PEG-lipid can have a linear or branched structure.

As used herein, the terms “molar percentage” and “mol %” refer to the number of moles of a given lipid component of a liposome divided by the total number of moles of all lipid components. Unless explicitly stated, the amounts of active agents, diluents, or other components are not included when calculating the mol % for a lipid component of a liposome.

As used herein, the term “loading” refers to effecting the accumulation of elinafide in a liposome. Elinafide can be encapsulated in the aqueous interior of the liposome, or it can be embedded in the lipid bilayer. Liposomes can be passively loaded, wherein the elinafide is included in the solution(s) used during liposome preparation. Alternatively, liposomes can be remotely loaded by establishing a chemical gradient (e.g., a pH or ion gradient) across the liposome bilayer, causing migration of the elinafide from the aqueous exterior to the liposome interior.

As used herein, the term “insertion” refers to the embedding of a lipid component into a liposome bilayer. In general, an amphiphilic lipid such as a PEG-lipid is transferred from solution to the bilayer due to van der Waals interactions between the hydrophobic portion of the amphiphilic lipid and the hydrophobic interior of the bilayer.

As used herein, the term “composition” refers to a product comprising the specified ingredients in the specified amounts, as well as any product which results, directly or indirectly, from combination of the specified ingredients in the specified amounts. Pharmaceutical compositions of the present invention generally contain a liposomal elinafide as described herein and a pharmaceutically acceptable carrier, diluent, or excipient. By “pharmaceutically acceptable,” it is meant that the carrier, diluent, or excipient must be compatible with the other ingredients of the formulation and non-deleterious to the recipient thereof.

As used herein, the term “cancer” refers to conditions including human cancers and carcinomas, sarcomas, adenocarcinomas, lymphomas, leukemias, and solid and lymphoid cancers. Examples of different types of cancer include, but are not limited to, lung cancer (e.g., non-small cell lung cancer or NSCLC), ovarian cancer, prostate cancer, colorectal cancer, liver cancer (i.e., hepatocarcinoma), renal cancer (i.e., renal cell carcinoma), bladder cancer, breast cancer, thyroid cancer, pleural cancer, pancreatic cancer, uterine cancer, cervical cancer, testicular cancer, anal cancer, pancreatic cancer, bile duct cancer, gastrointestinal carcinoid tumors, esophageal cancer, gall bladder cancer, appendix cancer, small intestine cancer, stomach (gastric) cancer, cancer of the central nervous system, cancer of unknown primary origin, skin cancer, choriocarcinoma, head and neck cancer, blood cancer, osteogenic sarcoma, fibrosarcoma, neuroblastoma, glioma, melanoma, B-cell lymphoma, non-Hodgkin's lymphoma, Burkitt's lymphoma, Small Cell lymphoma, Large Cell lymphoma, monocytic leukemia, myelogenous leukemia, acute lymphocytic leukemia, acute myelocytic leukemia, and multiple myeloma.

As used herein, the terms “treat,” “treating,” and “treatment” refer to any indicia of success in the treatment or amelioration of a cancer or a symptom of cancer, including any objective or subjective parameter, such as abatement, remission (e.g. full or partial), achieving a complete response in a patient, achieving a partial response in a patient, maintaining a stable disease state (e.g., the target lesions have not decreased in size, however, the target lesions have also not increased in size and new lesions have not formed), diminishing of symptoms or making the cancer or cancer symptom more tolerable to the patient (clinical benefit). The treatment or amelioration of symptoms can be based on any objective or subjective parameter, including, e.g., the result of a physical examination (clinical benefit) or clinical test.

As used herein, the terms “administer,” “administered,” or “administering” refer to methods of administering the liposome compositions of the present invention. The liposome compositions of the present invention can be administered in a variety of ways, including parenterally, intravenously, intradermally, intramuscularly, or intraperitoneally. The liposome compositions can also be administered as part of a composition or formulation.

As used herein, the term “subject” refers to any mammal, in particular a human, at any stage of life.

The term “half-life” or “t_{1/2}” as used herein refers to the amount of time required for the concentration or amount of the drug found in the blood or plasma to decrease by one-half. This decrease in drug concentration is a reflection of its metabolism plus excretion or elimination after absorption is complete and distribution has reached an equilibrium or quasi equilibrium state. The half-life of a drug in the blood may be determined graphically off of a pharmacokinetic plot of a drug's blood-concentration time plot, typically after intravenous administration to a sample population. The half-life can also be determined using mathematical calculations that are well known in the art. Further, as used herein the term “half-life” also includes the “apparent half-life” of a drug. The apparent half-life may be a composite number that accounts for contributions from other processes besides elimination, such as absorption, reuptake, or enterohepatic recycling.

The term “AUC” means an area under the drug concentration-time curve.

The term “Partial AUC” means an area under the drug concentration-time curve (AUC) calculated using linear trapezoidal summation for a specified interval of time, for example, AUC₍₀₋₁ₕᵣ₎, AUC₍₀₋₂ₕᵣ₎, AUC₍₀₋₄ₕᵣ₎, AUC₍₀₋₆ₕᵣ₎, AUC₍₀₋₈ₕᵣ₎, AUC_{(0-(Tmax of IR product+2SD))}, AUC₍₀₋₍ₓ₎ₕᵣ₎, AUC_{(x-yhr)}, AUC_{(Tmax-t)}, AUC₀₋₍ₜ₎ₕᵣ₎, AUC_{(Tmax of IR poduct+2SD)-t)}, or AUC_{(0-∞)}.

The term “Cm.” refers to the maximum plasma concentration obtain during a dosing interval.

The use of individual numerical values are stated as approximations as though the values were preceded by the word “about” or “approximately.” Similarly, the numerical values in the various ranges specified in this application, unless expressly indicated otherwise, are stated as approximations as though the minimum and maximum values within the stated ranges were both preceded by the word “about” or “approximately.” In this manner, variations above and below the stated ranges can be used to achieve substantially the same results as values within the ranges. As used herein, the terms “about” and “approximately” when referring to a numerical value shall have their plain and ordinary meanings to a person of ordinary skill in the art to which the disclosed subject matter is most closely related or the art relevant to the range or element at issue. The amount of broadening from the strict numerical boundary depends upon many factors. For example, some of the factors which may be considered include the criticality of the element and/or the effect a given amount of variation will have on the performance of the claimed subject matter, as well as other considerations known to those of skill in the art. As used herein, the use of differing amounts of significant digits for different numerical values is not meant to limit how the use of the words “about” or “approximately” will serve to broaden a particular numerical value or range. Thus, as a general matter, “about” or “approximately” broaden the numerical value. Also, the disclosure of ranges is intended as a continuous range including every value between the minimum and maximum values plus the broadening of the range afforded by the use of the term “about” or “approximately.” Consequently, recitation of ranges of values herein are merely intended to serve as a shorthand method of referring individually to each separate value falling within the range, unless otherwise indicated herein, and each separate value is incorporated into the specification as if it were individually recited herein.

### III. Embodiments of the Invention

In a first aspect, the present invention provides a liposome containing a phosphatidylcholine lipid, a sterol, PEG-lipid, and elinafide. In a further aspect, less than about 20% of elinafide is released in vitro from the liposome within about 40 hours.

In a second aspect, the present invention provides a liposomal elinafide composition for the treatment of cancer. The composition includes a liposome containing a phosphatidylcholine lipid, a sterol, PEG-lipid, and elinafide, and a pharmaceutically acceptable excipient.

In a third aspect, the present invention provides a method for preparing liposomal elinafide. The method includes: a) forming a first liposome having a lipid bilayer including a phosphatidylcholine lipid and a sterol, wherein the lipid bilayer encapsulates an interior compartment or space containing an aqueous solution; b) loading the first liposome with elinafide, or a pharmaceutically acceptable salt thereof, to form a loaded liposome; and, optionally c) forming a mixture containing the loaded liposome and a PEG-lipid under conditions sufficient to allow insertion of the PEG-lipid into the lipid bilayer.

In a fourth aspect, the present invention provides a method for treating cancer comprising administering to a patient in need thereof the liposomal elinafide composition of the invention. In a further aspect, the method for treating cancer has reduced incidence of muscle myopathy.

### Elinafide

Elinafide is a bis-naphthalimide whose structure is shown below.

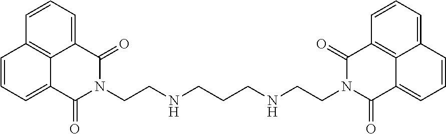 [SMILES: O=C1c2cccc3cccc(c23)C(=O)N1CCNCCCNCCN1C(=O)c2cccc3cccc(c23)C1=O]

In one embodiment, the present invention provides a liposome containing elinafide or a pharmaceutically acceptable salt thereof. In a further embodiment, the present invention provides a liposomal elinafide composition comprising the elinafide liposome and a pharmaceutically acceptable excipient.

Liposomal elinafide is surprisingly unique among naphthalimides and azonifides. Liposomal elinafide has been shown to be superior in all respects to free elinafide in both anti-tumor efficacy and tolerance (i.e., dose-limiting toxicity). Other naphthalimides and azonifides investigated herein, although successfully encapsulated into liposomes, have not shown the superior performance as compared to their free drug counterparts.

### Liposomes

The liposomes of the present invention can contain any suitable lipid, including cationic lipids, zwitterionic lipids, neutral lipids, or anionic lipids as described above. Suitable lipids can include fats, waxes, steroids, cholesterol, fat-soluble vitamins, monoglycerides, diglycerides, phospholipids, sphingolipids, glycolipids, cationic or anionic lipids, derivatized lipids, and the like.

In general, the liposomes of the present invention contain at least one phosphatidylcholine (PC) lipid. Suitable PC lipids include saturated PCs and unsaturated PCs.

Examples of saturated PCs include, but are not limited to, 1,2-dilauroyl-sn-glycero-3-phosphocholine (DLPC), 1,2-dimyristoyl-sn-glycero-3-phosphocholine (dimyristoylphosphatidylcholine; DMPC), 1,2-distearoyl-sn-glycero-3-phosphocholine (distearoylphosphatidylcholine; DSPC), 1,2-dioleoyl-sn-glycero-3-phosphocholine (DOPC), 1,2-dipalmitoyl-sn-glycero-3-phosphocholine (dipalmitoylphosphatidylcholine; DPPC), 1-myristoyl-2-palmitoyl-sn-glycero-3-phosphocholine (MPPC), 1-palmitoyl-2-myristoyl-sn-glycero-3-phosphocholine (PMPC), 1-myristoyl-2-stearoyl-sn-glycero-3-phosphocholine (MSPC), 1-palmitoyl-2-stearoyl-sn-glycero-3-phosphocholine (PSPC), 1-stearoyl-2-palmitoyl-sn-glycero-3-phosphocholine (SPPC), and 1-stearoyl-2-myristoyl-sn-glycero-3-phosphocholine (SMPC).

Examples of unsaturated PCs include, but are not limited to, 1,2-dimyristoleoyl-sn-glycero-3-phosphocholine, 1,2-dimyristelaidoyl-sn-glycero-3-phosphocholine, 1,2-dipamiltoleoyl-sn-glycero-3-phosphocholine, 1,2-dipalmitelaidoyl-sn-glycero-3-phosphocholine, 1,2-dioleoyl-sn-glycero-3-phosphocholine (DOPC), 1,2-dielaidoyl-sn-glycero-3-phosphocholine, 1,2-dipetroselenoyl-sn-glycero-3-phosphocholine, 1,2-dilinoleoyl-sn-glycero-3-phosphocholine, 1-palmitoyl-2-oleoyl-sn-glycero-3-phosphocholine (palmitoyloleoylphosphatidylcholine; POPC), 1-palmitoyl-2-linoleoyl-sn-glycero-3-phosphocholine, 1-stearoyl-2-oleoyl-sn-glycero-3-phosphocholine (SOPC), 1-stearoyl-2-linoleoyl-sn-glycero-3-phosphocholine, 1-oleoyl-2-myristoyl-sn-glycero-3-phosphocholine (OMPC), 1-oleoyl-2-palmitoyl-sn-glycero-3-phosphocholine (OPPC), and 1-oleoyl-2-stearoyl-sn-glycero-3-phosphocholine (OSPC).

Lipid extracts, such as egg PC, heart extract, brain extract, liver extract, soy PC, and hydrogenated soy PC (HSPC) are also useful in the present invention.

The compositions provided herein will, in some embodiments, consist essentially of PC lipid/cholesterol mixtures (with elinafide and PEG-lipid as described below). In some embodiments, the liposome compositions will consist essentially of a PC lipid or mixture of PC lipids, with cholesterol, a PEG-lipid, and elinafide. In still other embodiments, the liposome compositions will consist essentially of a single type of phosphatidylcholine lipid, with cholesterol, a PEG-lipid and elinafide. In some embodiments, when a single type of PC lipid is used, it is selected from DOPC, DSPC, HSPC, DPPC, POPC and SOPC.

In some embodiments, the PC lipid is selected from the group consisting of DPPC, DSPC, HSPC, and mixtures thereof. In some embodiments, the compositions of the present invention include liposomes containing about 55 to about 65 mol % of a PC lipid or mixture of PC lipids, or about 50 to about 65 mol % of a PC lipid or mixture of PC lipids, or about 45 to about 70 mol % of a PC lipid or mixture of PC lipids. The liposomes can contain, for example, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, or about 70 mol % of a PC lipid. In some embodiments, the liposomes contain about 56 mol % of a PC lipid. In still other embodiments, the liposomes contain about 55 mol % of a PC lipid. In additional embodiments, the liposomes contain about 54 mol % of a PC lipid. In further embodiments, the liposomes contain about 53 mol % of a PC lipid.

Other suitable phospholipids, generally used in low amounts or in amounts less than the PC lipids, include phosphatidic acids (PAs), phosphatidylethanolamines (PEs), phosphatidylglycerols (PGs), phosphatidylserine (PSs), and phosphatidylinositol (PIs). Examples of phospholipids include, but are not limited to, dimyristoylphosphatidylglycerol (DMPG), distearoylphosphatidylglycerol (DSPG), dioleoylphosphatidylglycerol (DOPG), dipalmitoylphosphatidylglycerol (DPPG), dimyristoylphosphatidylserine (DMPS), distearoylphosphatidylserine (DSPS), dioleoylphosphatidylserine (DOPS), dipalmitoylphosphatidylserine (DPPS), dioleoylphosphatidylethanolamine (DOPE), palmitoyloleoylphosphatidylcholine (POPC), palmitoyloleoylphosphatidylethanolamine (POPE), dipalmitoylphosphatidylethanolamine (DPPE), dimyristoylphosphoethanolamine (DMPE), distearoylphosphatidylethanolamine (DSPE), 16-O-monomethyl PE, 16-O-dimethyl PE, 18-1-trans PE, 1-stearoyl-2-oleoyl-phosphatidyethanolamine (SOPE), dielaidoylphosphoethanolamine (transDOPE), and cardiolipin.

In some embodiments, phospholipids can include reactive functional groups for further derivatization. Examples of such reactive lipids include, but are not limited to, dioleoylphosphatidylethanolamine-4-(N-maleimidomethyl)-cyclohexane-1-carboxylate (DOPE-mal) and dipalmitoylphosphatidylethanolamine-N-succinyl (succinyl-PE).

Liposomes of the present invention can contain steroids, characterized by the presence of a fused, tetracyclic gonane ring system. Examples of steroids include, but are not limited to, cholic acid, progesterone, cortisone, aldosterone, testosterone, dehydroepiandrosterone, and sterols such as estradiol and cholesterol. Synthetic steroids and derivatives thereof are also contemplated for use in the present invention.

In general, the liposomes contain at least one sterol. In some embodiments, the sterol is cholesterol (i.e., 2,15-dimethyl-14-(1,5-dimethylhexyl)tetracyclo[8.7.0.0^{2,7}.0^{11,15}]heptacos-7-en-5-ol). In some embodiments, the liposomes can contain about 30 to about 50 mol % of cholesterol, or about 35 to about 50 mol % of cholesterol, or about 30 to about 45 mol % of cholesterol. The liposomes can contain, for example, about 30, about 31, about 32, about 33, about 34, about 35, about 36, about 37, about 38, about 39, about 40, about 41, about 42, about 43, about 44, about 45, about 46, about 47, about 48, about 49, or about 50 mol % of cholesterol. In some embodiments, the liposomes contain about 30 and about 40 mol % of cholesterol. In some embodiments, the liposomes contain about 40 to about 45 mol % of cholesterol. In some embodiments, the liposomes contain about 45 mol % of cholesterol. In some embodiments, the liposomes contain about 44 mol % of cholesterol.

The liposomes of the present invention can include any suitable PEG-lipid derivative. In some embodiments, the PEG-lipid is a diacyl-phosphatidylethanolamine-N-[methoxy(polyethene glycol)]. The molecular weight of the poly(ethylene glycol) in the PEG-lipid is generally in the range of from about 500 Da to about 5000 Da. The PEG can have a molecular weight of, for example, about 750 Da, about 1000 Da, about 2000 Da, or about 5000 Da. In some embodiments, the PEG-lipid is selected from distearoyl-phosphatidylethanolamine-N-[methoxy(polyethene glycol)-2000] (DSPE-PEG-2000) and distearoyl-phosphatidylethanolamine-N-[methoxy(polyethene glycol)-5000] (DSPE-PEG-5000). In some embodiments, the PEG-lipid is DSPE-PEG-2000.

In general, the compositions of the present invention include liposomes containing about 2 to about 8 mol % of a PEG-lipid. The liposomes can contain, for example, about 2, about 3, about 4, about 5, about 6, about 7, or about 8 mol % of a PEG-lipid. In some embodiments, the liposomes contain about 2 to about 6 mol % of a PEG-lipid. In some embodiments, the liposomes contain about 3 mol % of a PEG-lipid. In some embodiments, the liposomes contain about 3 mol % of DSPE-PEG-2000.

The liposomes of the present invention can also include some amounts of cationic lipids, which are generally amounts lower than the amount of phosphatidylcholine lipid. Cationic lipids contain positively charged functional groups under physiological conditions. Cationic lipids include, but are not limited to, N,N-dioleyl-N,N-dimethylammonium chloride (DODAC), N,N-distearyl-N,N-dimethylammonium bromide (DDAB), N-(1-(2,3-dioleoyloxy)propyl)-N,N,N-trimethylammonium chloride (DOTAP), N-(1-(2,3-dioleyloxy)propyl)-N,N,N-trimethylammonium chloride (DOTMA), N-[1-(2,3,-ditetradecyloxy)propyl]-N,N-dimethyl-N-hydroxyethylammonium bromide (DMRIE), N-[1-(2,3,dioleyloxy)propyl]-N,N-dimethyl-N-hydroxy ethylammonium bromide (DORIE), 3β-[N—(N′,N′-dimethylaminoethane) carbamoyl]cholesterol (DC-Chol), dimethyldioctadecylammonium (DDAB), and N,N-dimethyl-2,3-dioleyloxy)propylamine (DODMA).

In some embodiments of the present invention, the liposome includes from about 50 mol % to about 70 mol % of DSPC and from about 25 mol % to about 45 mol % of cholesterol. In some embodiments, the liposome includes about 53 mol % of DSPC, about 44 mol % of cholesterol, and about 3 mol % of DSPE-PEG-2000. In some embodiments, the liposome includes about 66 mol % of DSPC, about 30 mol % of cholesterol, and about 4 mol % of DSPE-PEG-2000.

### Diagnostic Agents

The liposomes of the present invention may also contain diagnostic agents. A diagnostic agent used in the present invention can include any diagnostic agent known in the art, as provided, for example, in the following references: Armstrong et al., Diagnostic Imaging, 5th Ed., Blackwell Publishing (2004); Torchilin, V. P., Ed., Targeted Delivery of Imaging Agents, CRC Press (1995); Vallabhajosula, S., Molecular Imaging. Radiopharmaceuticals for PET and SPECT, Springer (2009). A diagnostic agent can be detected by a variety of ways, including as an agent providing and/or enhancing a detectable signal that includes, but is not limited to, gamma-emitting, radioactive, echogenic, optical, fluorescent, absorptive, magnetic, or tomography signals. Techniques for imaging the diagnostic agent can include, but are not limited to, single photon emission computed tomography (SPECT), magnetic resonance imaging (MRI), optical imaging, positron emission tomography (PET), computed tomography (CT), x-ray imaging, gamma ray imaging, and the like. The diagnostic agents can be associated with the therapeutic liposome in a variety of ways, including for example being embedded or encapsulated in the liposome.

In some embodiments, a diagnostic agent can include chelators that bind to metal ions to be used for a variety of diagnostic imaging techniques. Exemplary chelators include, but are not limited to, ethylenediaminetetraacetic acid (EDTA), [4-(1,4,8, 11-tetraazacyclotetradec-1-yl) methyl]benzoic acid (CPTA), cyclohexanediaminetetraacetic acid (CDTA), ethylenebis(oxyethylenenitrilo)tetraacetic acid (EGTA), diethylenetriaminepentaacetic acid (DTPA), citric acid, hydroxyethyl ethylenediamine triacetic acid (HEDTA), iminodiacetic acid (IDA), triethylene tetraamine hexaacetic acid (TTHA), 1,4,7, 10-tetraazacyclododecane-1,4,7,10-tetra(methylene phosphonic acid) (DOTP), 1,4,8,11-tetraazacyclododecane-1,4,8,11-tetraacetic acid (TETA), 1,4,7,10-tetraazacyclododecane-1,4,7,10-tetraacetic acid (DOTA), and derivatives thereof.

A radioisotope can be incorporated into some of the diagnostic agents described herein and can include radionuclides that emit gamma rays, positrons, beta and alpha particles, and X-rays. Suitable radionuclides include but are not limited to ²²⁵Ac, ⁷²As, ²¹¹At, ¹¹B, ¹²⁸Ba, ²¹²Bi, ⁷⁵Br, ⁷⁷Br, ¹⁴C, ¹⁰⁹Cd, ⁶²Cu, ⁶⁴Cu, ⁶⁷Cu, ¹⁸F, ⁶⁷Ga, ⁶⁸Ga ³H, ¹²³I, ¹²⁵I, ¹³⁰I, ¹³¹I, ¹¹¹In, ¹⁷⁷Lu, ¹³N, ¹⁵O, ³²P, ³³P, ²¹²Pb, ¹⁰³Pd, ¹⁸⁶Re, ¹⁸⁸Re, ⁴⁷Sc, ¹⁵³Sm, ⁸⁹Sr, ^{99m}Tc, ⁸⁸Y, and ⁹⁰Y. In certain embodiments, radioactive agents can include ¹¹¹In-DTPA, ^{99m}Tc(CO)₃-DTPA, ^{99m}Tc(CO)₃-ENPy2, ^{62/64/67}Cu-TETA, ^{99m}Tc(CO)₃-IDA, and ^{99m}Tc(CO)₃triamines (cyclic or linear). In other embodiments, the agents can include DOTA and its various analogs with ¹¹¹In, ¹⁷⁷Lu, ¹⁵³Sm, ^{88/90}Y, ^{62/64/67}Cu, or ^{67/68}Ga. In some embodiments, the liposomes can be radiolabeled, for example, by incorporation of lipids attached to chelates, such as DTPA-lipid, as provided in the following references: Phillips et al., Wiley Interdisciplinary Reviews: Nanomedicine and Nanobiotechnology, 1(1): 69-83 (2008); Torchilin, V. P. & Weissig, V., Eds. Liposomes 2nd Ed: Oxford Univ. Press (2003); Elbayoumi, T. A. & Torchilin, V. P., Eur. J. Nucl. Med. Mol. Imaging 33:1196-1205 (2006); Mougin-Degraef, M. et al., Int'l J. Pharmaceutics 344:110-117 (2007).

In other embodiments, the diagnostic agents can include optical agents such as fluorescent agents, phosphorescent agents, chemiluminescent agents, and the like. Numerous agents (e.g., dyes, probes, labels, or indicators) are known in the art and can be used in the present invention. (See, e.g., Invitrogen, The Handbook—A Guide to Fluorescent Probes and Labeling Technologies, Tenth Edition (2005)). Fluorescent agents can include a variety of organic and/or inorganic small molecules or a variety of fluorescent proteins and derivatives thereof. For example, fluorescent agents can include, but are not limited to, cyanines, phthalocyanines, porphyrins, indocyanines, rhodamines, phenoxazines, phenylxanthenes, phenothiazines, phenoselenazines, fluoresceins, benzoporphyrins, squaraines, dipyrrolo pyrimidones, tetracenes, quinolines, pyrazines, corrins, croconiums, acridones, phenanthridines, rhodamines, acridines, anthraquinones, chalcogenopyrylium analogues, chlorins, naphthalocyanines, methine dyes, indolenium dyes, azo compounds, azulenes, azaazulenes, triphenyl methane dyes, indoles, benzoindoles, indocarbocyanines, benzoindocarbocyanines, and BODIPY™ derivatives having the general structure of 4,4-difluoro-4-bora-3a,4a-diaza-s-indacene, and/or conjugates and/or derivatives of any of these. Other agents that can be used include, but are not limited to, for example, fluorescein, fluorescein-polyaspartic acid conjugates, fluorescein-polyglutamic acid conjugates, fluorescein-polyarginine conjugates, indocyanine green, indocyanine-dodecaaspartic acid conjugates, indocyanine-polyaspartic acid conjugates, isosulfan blue, indole disulfonates, benzoindole disulfonate, bis(ethylcarboxymethyl)indocyanine, bis(pentylcarboxymethyl)indocyanine, polyhydroxyindole sulfonates, polyhydroxybenzoindole sulfonate, rigid heteroatomic indole sulfonate, indocyaninebispropanoic acid, indocyaninebishexanoic acid, 3,6-dicyano-2,5-[(N,N,N′,N′-tetrakis(carboxymethyl)amino]pyrazine, 3,6-[(N,N,N′,N′-tetrakis(2-hydroxyethyl)amino]pyrazine-2,5-dicarboxylic acid, 3,6-bis(N-azatedino)pyrazine-2,5-dicarboxylic acid, 3,6-bis(N-morpholino)pyrazine-2,5-dicarboxylic acid, 3,6-bis(N-piperazino)pyrazine-2,5-dicarboxylic acid, 3,6-bis(N-thiomorpholino)pyrazine-2,5-dicarboxylic acid, 3,6-bis(N-thiomorpholino)pyrazine-2,5-dicarboxylic acid S-oxide, 2,5-dicyano-3,6-bis(N-thiomorpholino)pyrazine S,S-dioxide, indocarbocyaninetetrasulfonate, chloroindocarbocyanine, and 3,6-diaminopyrazine-2,5-dicarboxylic acid.

One of ordinary skill in the art will appreciate that particular optical agents used can depend on the wavelength used for excitation, depth underneath skin tissue, and other factors generally well known in the art. For example, optimal absorption or excitation maxima for the optical agents can vary depending on the agent employed, but in general, the optical agents of the present invention will absorb or be excited by light in the ultraviolet (UV), visible, or infrared (IR) range of the electromagnetic spectrum. For imaging, dyes that absorb and emit in the near-IR (˜700-900 nm, e.g., indocyanines) are preferred. For topical visualization using an endoscopic method, any dyes absorbing in the visible range are suitable.

In some embodiments, the non-ionizing radiation employed in the process of the present invention can range in wavelength from about 350 nm to about 1200 nm. In one exemplary embodiment, the fluorescent agent can be excited by light having a wavelength in the blue range of the visible portion of the electromagnetic spectrum (from about 430 nm to about 500 nm) and emits at a wavelength in the green range of the visible portion of the electromagnetic spectrum (from about 520 nm to about 565 nm). For example, fluorescein dyes can be excited with light with a wavelength of about 488 nm and have an emission wavelength of about 520 nm. As another example, 3,6-diaminopyrazine-2,5-dicarboxylic acid can be excited with light having a wavelength of about 470 nm and fluoresces at a wavelength of about 532 nm. In another embodiment, the excitation and emission wavelengths of the optical agent may fall in the near-infrared range of the electromagnetic spectrum. For example, indocyanine dyes, such as indocyanine green, can be excited with light with a wavelength of about 780 nm and have an emission wavelength of about 830 nm.

In yet other embodiments, the diagnostic agents can include, but are not limited to, magnetic resonance (MR) and x-ray contrast agents that are generally well known in the art, including, for example, iodine-based x-ray contrast agents, superparamagnetic iron oxide (SPIO), complexes of gadolinium or manganese, and the like. (See, e.g., Armstrong et al., Diagnostic Imaging, 5th Ed., Blackwell Publishing (2004)). In some embodiments, a diagnostic agent can include a MR imaging agent. Exemplary MR agents include, but are not limited to, paramagnetic agents, superparamagnetic agents, and the like. Exemplary paramagnetic agents can include, but are not limited to, gadopentetic acid, gadoteric acid, gadodiamide, gadolinium, gadoteridol, mangafodipir, gadoversetamide, ferric ammonium citrate, gadobenic acid, gadobutrol, or gadoxetic acid. Superparamagnetic agents can include, but are not limited to, superparamagnetic iron oxide and ferristene. In certain embodiments, the diagnostic agents can include x-ray contrast agents as provided, for example, in the following references: H. S Thomsen, R. N. Muller and R. F. Mattrey, Eds., Trends in Contrast Media, (Berlin: Springer-Verlag, 1999); P. Dawson, D. Cosgrove and R. Grainger, Eds., Textbook of Contrast Media (ISIS Medical Media 1999); Torchilin, V. P., Curr. Pharm. Biotech. 1:183-215 (2000); Bogdanov, A. A. et al., Adv. Drug Del. Rev. 37:279-293 (1999); Sachse, A. et al., Investigative Radiology 32(1):44-50 (1997). Examples of x-ray contrast agents include, without limitation, iopamidol, iomeprol, iohexol, iopentol, iopromide, iosimide, ioversol, iotrolan, iotasul, iodixanol, iodecimol, ioglucamide, ioglunide, iogulamide, iosarcol, ioxilan, iopamiron, metrizamide, iobitridol, and iosimenol. In certain embodiments, the x-ray contrast agents can include iopamidol, iomeprol, iopromide, iohexol, iopentol, ioversol, iobitridol, iodixanol, iotrolan, and iosimenol.

### Targeting Agents

In some embodiments, liposome accumulation at a target site may be due to the enhanced permeability and retention characteristics of certain tissues such as cancer tissues. Accumulation in such a manner often results in part because of liposome size and may not require special targeting functionality. In other embodiments, the liposomes of the present invention can also include a targeting agent. Generally, the targeting agents of the present invention can associate with any target of interest, such as a target associated with an organ, tissues, cell, extracellular matrix, or intracellular region. In certain embodiments, a target can be associated with a particular disease state, such as a cancerous condition. In some embodiments, the targeting component can be specific to only one target, such as a receptor. Suitable targets can include, but are not limited to, a nucleic acid, such as a DNA, RNA, or modified derivatives thereof. Suitable targets can also include, but are not limited to, a protein, such as an extracellular protein, a receptor, a cell surface receptor, a tumor-marker, a transmembrane protein, an enzyme, or an antibody. Suitable targets can include a carbohydrate, such as a monosaccharide, disaccharide, or polysaccharide that can be, for example, present on the surface of a cell.

In certain embodiments, a targeting agent can include a target ligand (e.g., an RGD-containing peptide), a small molecule mimic of a target ligand (e.g., a peptide mimetic ligand), or an antibody or antibody fragment specific for a particular target. In some embodiments, a targeting agent can further include folic acid derivatives, B-12 derivatives, integrin RGD peptides, NGR derivatives, somatostatin derivatives or peptides that bind to the somatostatin receptor, e.g., octreotide and octreotate, and the like. The targeting agents of the present invention can also include an aptamer. Aptamers can be designed to associate with or bind to a target of interest. Aptamers can be comprised of, for example, DNA, RNA, and/or peptides, and certain aspects of aptamers are well known in the art. (See. e.g., Klussman, S., Ed., The Aptamer Handbook, Wiley-VCH (2006); Nissenbaum, E. T., Trends in Biotech. 26(8): 442-449 (2008)).

### Methods for Preparing Liposomal Elinafide

The present invention provides methods for preparing a liposomal elinafide. Liposomes can be prepared and loaded with elinafide using a number of techniques that are known to those of skill in the art. Lipid vesicles can be prepared, for example, by hydrating a dried lipid film (prepared via evaporation of a mixture of the lipid and an organic solvent in a suitable vessel) with water or an aqueous buffer. Hydration of lipid films typically results in a suspension of multilamellar vesicles (MLVs). Alternatively, MLVs can be formed by diluting a solution of a lipid in a suitable solvent, such as a C₁₋₄ alkanol, with water or an aqueous buffer. Unilamellar vesicles can be formed from MLVs via sonication or extrusion through membranes with defined pore sizes. Encapsulation of elinafide can be conducted by including the drug in the aqueous solution used for film hydration or lipid dilution during MLV formation. Elinafide can also be encapsulated in pre-formed vesicles using “remote loading” techniques. Remote loading includes the establishment of a pH- or ion-gradient on either side of the vesicle membrane, which drives elinafide from the exterior solution to the interior of the vesicle.

Accordingly, some embodiments of the present invention provide a method for preparing a liposomal elinafide including: a) forming a first liposome having a lipid bilayer including a phosphatidylcholine lipid and a sterol, wherein the lipid bilayer encapsulates an interior compartment or space containing an aqueous solution; b) loading the first liposome with elinafide, or a pharmaceutically acceptable salt thereof, to form a loaded liposome.

The lipids used in the methods of the invention are generally as described above. However, the route to the liposomal elinafide will depend in part on the identity of the lipids and the quantities and combinations that are used. For example, elinafide can be encapsulated in vesicles at various stages of liposome preparation. In some embodiments, the first liposome is formed such that the lipid bilayer comprises DSPC and cholesterol, and the DSPC:cholesterol ratio is about 55:45 (mol:mol). In another embodiment, the first liposome is formed such that the lipid bilayer comprises DSPC and cholesterol, and the DSPC:cholesterol ratio is about 70:30 (mol:mol). In still another embodiment, the interior of the first liposome contains aqueous ammonium sulfate or citrate buffer. Loading the first liposomes can include forming an aqueous solution containing the first liposome and elinafide or pharmaceutically acceptable salt thereof under conditions sufficient to allow accumulation of elinafide in the interior compartment of the first liposome.

Loading conditions generally include a higher ammonium sulfate or citrate concentration in the interior of the first liposome than in the exterior aqueous solution. In some embodiments, the loading step is conducted at a temperature above the gel-to-fluid phase transition temperature (Tₘ) of one or more of the lipid components in the liposomes. The loading can be conducted, for example, at about 50° C., about 55° C., about 60° C., about 65° C., or at about 70° C. In some embodiments, the loading step is conducted at a temperature of from about 50° C. to about 70° C. Loading can be conducted using any suitable amount of elinafide. In general, elinafide is used in an amount such that the ratio of the combined weight of the phosphatidylcholine and the sterol in the liposome to the weight of elinafide is from about 1:0.01 to about 1:1. The ratio of the combined phosphatidylcholine/sterol to the weight of elinafide can be, for example, about 1:0.01, about 1:0.05, about 1:0.10, about 1:0.15, about 1:0.20, about 1:0.25, about 1:0.30, about 1:0.35, about 1:0.40, about 1:0.45, about 1:0.50, about 1:0.55, about 1:0.60, about 1:0.65, about 1:0.70, about 1:0.75, about 1:0.80, about 1:0.85, about 1:0.90, about 1:0.95, or about 1:1. In some embodiments, the loading step is conducted such that the ratio of the combined weight of the phosphatidylcholine and the sterol to the weight of elinafide is from about 1:0.01 to about 1:1. In some embodiments, the ratio of the combined weight of the phosphatidylcholine and the sterol to the weight of elinafide is from about 1:0.05 to about 1:0.5. In some embodiments, the ratio of the combined weight of the phosphatidylcholine and the sterol to the weight of elinafide is about 1:0.2. The loading step can be conducted for any amount of time that is sufficient to allow accumulation of elinafide in the liposome interior at a desired level.

The PEG-lipid can also be incorporated into lipid vesicles at various stages of the liposome preparation. For example, MLVs containing a PEG-lipid can be prepared prior to loading with elinafide. Alternatively, a PEG-lipid can be inserted into a lipid bilayer after loading of a vesicle with elinafide. The PEG-lipid can be inserted into MLVs prior to extrusion of SUVs, or the PEG-lipid can be inserted into pre-formed SUVs.

Accordingly, some embodiments of the invention provide a method for preparing a liposomal elinafide wherein the method includes: a) forming a first liposome having a lipid bilayer including a phosphatidylcholine lipid and a sterol, wherein the lipid bilayer encapsulates an interior compartment comprising an aqueous solution; b) loading the first liposome with elinafide, or a pharmaceutically acceptable salt thereof, to form a loaded liposome; and c) forming a mixture containing the loaded liposome and a PEG-lipid under conditions sufficient to allow insertion of the PEG-lipid into the lipid bilayer.

In some embodiments, the insertion of the PEG-lipid is conducted at a temperature of from about 35° C. to about 70° C. The loading can be conducted, for example, at about 35° C., about 40° C., about 45° C., about 50° C., about 55° C., about 60° C., about 65° C., or at about 70° C. In some embodiments, insertion of the PEG-lipid is conducted at a temperature of from about 50° C. to about 55° C. Insertion can be conducted using any suitable amount of the PEG-lipid. In general, the PEG-lipid is used in an amount such that the ratio of the combined number of moles of the phosphatidylcholine and the sterol to the number of moles of the PEG-lipid is from about 1000:1 to about 20:1. The molar ratio of the combined phosphatidylcholine/sterol to PEG lipid can be, for example, about 1000:1, about 950:1, about 900:1, about 850:1, about 800:1, about 750:1, about 700:1, about 650:1, about 600:1, about 550:1, about 500:1, about 450:1, about 400:1, about 350:1, about 300:1, about 250:1, about 200:1, about 150:1, about 100:1, about 50:1, or about 20:1. In some embodiments, the loading step is conducted such that the ratio of combined phosphatidylcholine and sterol to PEG-lipid is from about 1000:1 to about 20:1 (mol:mol). In some embodiments, the ratio of the combined phosphatidylcholine and sterol to the PEG-lipid is from about 100:1 to about 20:1 (mol:mol). In some embodiments, the ratio of the combined phosphatidylcholine and sterol to the PEG-lipid is from about 35:1 (mol:mol) to about 25:1 (mol:mol). In some embodiments, the ratio of the combined phosphatidylcholine and sterol to the PEG-lipid is about 33:1 (mol:mol). In some embodiments, the ratio of the combined phosphatidylcholine and sterol to the PEG-lipid is about 27:1 (mol:mol).

A number of additional preparative techniques known to those of skill in the art can be included in the methods of the invention. Liposomes can be exchanged into various buffers by techniques including dialysis, size exclusion chromatography, diafiltration, and ultrafiltration. Buffer exchange can be used to remove unencapsulated elinafide and other unwanted soluble materials from the compositions. Aqueous buffers and certain organic solvents can be removed from the liposomes via lyophilization. In some embodiments, the methods of the invention include exchanging the liposomal elinafide from the mixture in step c) to an aqueous solution that is substantially free of unencapsulated elinafide and uninserted PEG-lipid. In some embodiments, the methods include lyophilizing the liposomal elinafide.

### Methods of Treating Cancer

In another aspect, the invention provides a method of treating cancer. The method includes administering to a subject in need thereof a composition containing a liposomal elinafide as described above. In therapeutic use for the treatment of cancer, the liposome compositions of the present invention can be administered such that the initial dosage of elinafide ranges from about 0.001 mg/kg to about 1000 mg/kg daily. A daily dose of about 0.01 to about 500 mg/kg, or about 0.1 to about 200 mg/kg, or about 1 to about 100 mg/kg, or about 10 to about 50 mg/kg, or about 10 mg/kg, or about 5 mg/kg, or about 2.5 mg/kg, or about 1 mg/kg can be used. Further, a daily dose of 3, 6, 12, 24, 48, 80, 120, 160, 190, 225, 270, and 320 mg/M² can be used.

The dosages may be varied depending upon the requirements of the patient, the type and severity of the cancer being treated, and the liposome composition being employed. For example, dosages can be empirically determined considering the type and stage of cancer diagnosed in a particular patient. The dose administered to a patient should be sufficient to affect a beneficial therapeutic response in the patient over time. The size of the dose will also be determined by the existence, nature, and extent of any adverse side-effects that accompany the administration of a particular liposome composition in a particular patient. Determination of the proper dosage for a particular situation is within the skill of the practitioner. Generally, treatment is initiated with smaller dosages which are less than the optimum dose of the liposome composition. Thereafter, the dosage is increased by small increments until the optimum effect under circumstances is reached. For convenience, the total daily dosage may be divided and administered in portions during the day, if desired. The duration of the infusion may be extended and/or the infusion may be interrupted in the case of an adverse event, but the total duration of the infusion cannot exceed 2 hours and cannot be resumed for several hours following the initiation of the infusion.

The methods described herein apply especially to solid tumor cancers (solid tumors), which are cancers of organs and tissue (as opposed to hematological malignancies), and ideally epithelial cancers. Examples of solid tumor cancers include bladder cancer, breast cancer, cervical cancer, colorectal cancer (CRC), esophageal cancer, gastric cancer, head and neck cancer, hepatocellular cancer, lung cancer, melanoma, neuroendocrine cancer, ovarian cancer, pancreatic cancer, prostate cancer, renal cancer, and thymus cancer. In one group of embodiments, the solid tumor cancer suitable for treatment according to the methods of the invention is selected from CRC, breast cancer, and prostate cancer. In another group of embodiments, the methods of the invention apply to treatment of hematological malignancies, including, for example, multiple myeloma, T-cell lymphoma, B-cell lymphoma, Hodgkins disease, non-Hodgkins lymphoma, acute myeloid leukemia, and chronic myelogenous leukemia.

The methods described herein takes advantage of the sustained plasma levels of liposomal elinafide and the enhanced tumor penetration and retention (EPR) effect to improve therapeutic efficacy without inducing common side effect associated with free elinafide. The methods described herein reduce such side effects, including reduced incidence of muscle myopathy, myelosuppression, neuromuscular toxicity, and QTc prolongation.

The compositions may be administered alone in the methods of the invention, or in combination with other therapeutic agents. The additional agents can be anticancer agents belonging to several classes of drugs such as, but not limited to, cytotoxic agents, VEGF-inhibitors, tyrosine kinase inhibitors, monoclonal antibodies, and immunotherapies. Examples of such agents include, but are not limited to, doxorubicin, cisplatin, oxaliplatin, carboplatin, 5-fluorouracil, gemcitabine (anti-metabolite), ramucirumab (VEGF 2 inhibitor), bevacizumab, trastuzumab (monoclonal antibody HER2 inhibitor), afatinib (EGFR tyrosine kinase inhibitor) and others. Additional anti-cancer agents can include, but are not limited to, 20-epi-1,25 dihydroxyvitamin D3,4-ipomeanol, 5-ethynyluracil, 9-dihydrotaxol, abiraterone, acivicin, aclarubicin, acodazole hydrochloride, acronine, acylfulvene, adecypenol, adozelesin, aldesleukin, all-tk antagonists, altretamine, ambamustine, ambomycin, ametantrone acetate, amidox, amifostine, aminoglutethimide, aminolevulinic acid, amrubicin, amsacrine, anagrelide, anastrozole, andrographolide, angiogenesis inhibitors, antagonist D, antagonist G, antarelix, anthramycin, anti-dorsalizing morphogenetic protein-1, antiestrogen, antineoplaston, antisense oligonucleotides, aphidicolin glycinate, apoptosis gene modulators, apoptosis regulators, apurinic acid, ARA-CDP-DL-PTBA, arginine deaminase, asparaginase, asperlin, asulacrine, atamestane, atrimustine, axinastatin 1, axinastatin 2, axinastatin 3, azacitidine, azasetron, azatoxin, azatyrosine, azetepa, azotomycin, baccatin III derivatives, balanol, batimastat, benzochlorins, benzodepa, benzoylstaurosporine, beta lactam derivatives, beta-alethine, betaclamycin B, betulinic acid, BFGF inhibitor, bicalutamide, bisantrene, bisantrene hydrochloride, bisaziridinylspermine, bisnafide, bisnafide dimesylate, bistratene A, bizelesin, bleomycin, bleomycin sulfate, BRC/ABL antagonists, breflate, brequinar sodium, bropirimine, budotitane, busulfan, buthionine sulfoximine, cactinomycin, calcipotriol, calphostin C, calusterone, camptothecin derivatives, canarypox IL-2, capecitabine, caracemide, carbetimer, carboplatin, carboxamide-amino-triazole, carboxyamidotriazole, carest M3, carmustine, cam 700, cartilage derived inhibitor, carubicin hydrochloride, carzelesin, casein kinase inhibitors, castanospermine, cecropin B, cedefingol, cetrorelix, chlorambucil, chlorins, chloroquinoxaline sulfonamide, cicaprost, cirolemycin, cisplatin, cis-porphyrin, cladribine, clomifene analogs, clotrimazole, collismycin A, collismycin B, combretastatin A4, combretastatin analog, conagenin, crambescidin 816, crisnatol, crisnatol mesylate, cryptophycin 8, cryptophycin A derivatives, curacin A, cyclopentanthraquinones, cyclophosphamide, cycloplatam, cypemycin, cytarabine, cytarabine ocfosfate, cytolytic factor, cytostatin, dacarbazine, dacliximab, dactinomycin, daunorubicin hydrochloride, decitabine, dehydrodidemnin B, deslorelin, dexifosfamide, dexormaplatin, dexrazoxane, dexverapamil, dezaguanine, dezaguanine mesylate, diaziquone, didemnin B, didox, diethylnorspermine, dihydro-5-azacytidine, dioxamycin, diphenyl spiromustine, docetaxel, docosanol, dolasetron, doxifluridine, doxorubicin, doxorubicin hydrochloride, droloxifene, droloxifene citrate, dromostanolone propionate, dronabinol, duazomycin, duocarmycin SA, ebselen, ecomustine, edatrexate, edelfosine, edrecolomab, eflomithine, eflomithine hydrochloride, elemene, elsamitrucin, emitefur, enloplatin, enpromate, epipropidine, epirubicin, epirubicin hydrochloride, epristeride, erbulozole, erythrocyte gene therapy vector system, esorubicin hydrochloride, estramustine, estramustine analog, estramustine phosphate sodium, estrogen agonists, estrogen antagonists, etanidazole, etoposide, etoposide phosphate, etoprine, exemestane, fadrozole, fadrozole hydrochloride, fazarabine, fenretinide, filgrastim, finasteride, flavopiridol, flezelastine, floxuridine, fluasterone, fludarabine, fludarabine phosphate, fluorodaunorunicin hydrochloride, fluorouracil, fluorocitabine, forfenimex, formestane, fosquidone, fostriecin, fostriecin sodium, fotemustine, gadolinium texaphyrin, gallium nitrate, galocitabine, ganirelix, gelatinase inhibitors, gemcitabine, gemcitabine hydrochloride, glutathione inhibitors, hepsulfam, heregulin, hexamethylene bisacetamide, hydroxyurea, hypericin, ibandronic acid, idarubicin, idarubicin hydrochloride, idoxifene, idramantone, ifosfamide, ilmofosine, ilomastat, imidazoacridones, imiquimod, immunostimulant peptides, insulin-like growth factor-1 receptor inhibitor, interferon agonists, interferon alpha-2A, interferon alpha-2B, interferon alpha-N1, interferon alpha-N3, interferon beta-IA, interferon gamma-IB, interferons, interleukins, iobenguane, iododoxorubicin, iproplatin, irinotecan, irinotecan hydrochloride, iroplact, irsogladine, isobengazole, isohomohalicondrin B, itasetron, jasplakinolide, kahalalide F, lamellarin-N triacetate, lanreotide, lanreotide acetate, leinamycin, lenograstim, lentinan sulfate, leptolstatin, letrozole, leukemia inhibiting factor, leukocyte alpha interferon, leuprolide acetate, leuprolide/estrogen/progesterone, leuprorelin, levamisole, liarozole, liarozole hydrochloride, linear polyamine analog, lipophilic disaccharide peptide, lipophilic platinum compounds, lissoclinamide 7, lobaplatin, lombricine, lometrexol, lometrexol sodium, lomustine, lonidamine, losoxantrone, losoxantrone hydrochloride, lovastatin, loxoribine, lurtotecan, lutetium texaphyrin, lysofylline, lytic peptides, maitansine, mannostatin A, marimastat, masoprocol, maspin, matrilysin inhibitors, matrix metalloproteinase inhibitors, maytansine, mechlorethamine hydrochloride, megestrol acetate, melengestrol acetate, melphalan, menogaril, merbarone, mercaptopurine, meterelin, methioninase, methotrexate, methotrexate sodium, metoclopramide, metoprine, meturedepa, microalgal protein kinase C inhibitors, MIF inhibitor, mifepristone, miltefosine, mirimostim, mismatched double stranded RNA, mitindomide, mitocarcin, mitocromin, mitogillin, mitoguazone, mitolactol, mitomalcin, mitomycin, mitomycin analogs, mitonafide, mitosper, mitotane, mitotoxin fibroblast growth factor-saporin, mitoxantrone, mitoxantrone hydrochloride, mofarotene, molgramostim, monoclonal antibody, human chorionic gonadotrophin, monophosphoryl lipid a/myobacterium cell wall SK, mopidamol, multiple drug resistance gene inhibitor, multiple tumor suppressor 1-based therapy, mustard anticancer agent, mycaperoxide B, mycobacterial cell wall extract, mycophenolic acid, myriaporone, n-acetyldinaline, nafarelin, nagrestip, naloxone/pentazocine, napavin, naphterpin, nartograstim, nedaplatin, nemorubicin, neridronic acid, neutral endopeptidase, nilutamide, nisamycin, nitric oxide modulators, nitroxide antioxidant, nitrullyn, nocodazole, nogalamycin, n-substituted benzamides, 06-benzylguanine, octreotide, okicenone, oligonucleotides, onapristone, ondansetron, oracin, oral cytokine inducer, ormaplatin, osaterone, oxaliplatin, oxaunomycin, oxisuran, paclitaxel, paclitaxel analogs, paclitaxel derivatives, palauamine, palmitoylrhizoxin, pamidronic acid, panaxytriol, panomifene, parabactin, pazelliptine, pegaspargase, peldesine, peliomycin, pentamustine, pentosan polysulfate sodium, pentostatin, pentrozole, peplomycin sulfate, perflubron, perfosfamide, perillyl alcohol, phenazinomycin, phenylacetate, phosphatase inhibitors, picibanil, pilocarpine hydrochloride, pipobroman, piposulfan, pirarubicin, piritrexim, piroxantrone hydrochloride, placetin A, placetin B, plasminogen activator inhibitor, platinum complex, platinum compounds, platinum-triamine complex, plicamycin, plomestane, porfimer sodium, porfiromycin, prednimustine, procarbazine hydrochloride, propyl bis-acridone, prostaglandin J2, prostatic carcinoma antiandrogen, proteasome inhibitors, protein A-based immune modulator, protein kinase C inhibitor, protein tyrosine phosphatase inhibitors, purine nucleoside phosphorylase inhibitors, puromycin, puromycin hydrochloride, purpurins, pyrazofurin, pyrazoloacridine, pyridoxylated hemoglobin polyoxyethylene conjugate, RAF antagonists, raltitrexed, ramosetron, RAS farnesyl protein transferase inhibitors, RAS inhibitors, RAS-GAP inhibitor, retelliptine demethylated, rhenium RE 186 etidronate, rhizoxin, riboprine, ribozymes, RII retinamide, RNAi, rogletimide, rohitukine, romurtide, roquinimex, rubiginone B1, ruboxyl, safingol, safingol hydrochloride, saintopin, sarcnu, sarcophytol A, sargramostim, SDI 1 mimetics, semustine, senescence derived inhibitor 1, sense oligonucleotides, signal transduction inhibitors, signal transduction modulators, simtrazene, single chain antigen binding protein, sizofuran, sobuzoxane, sodium borocaptate, sodium phenylacetate, solverol, somatomedin binding protein, sonermin, sparfosate sodium, sparfosic acid, sparsomycin, spicamycin D, spirogermanium hydrochloride, spiromustine, spiroplatin, splenopentin, spongistatin 1, squalamine, stem cell inhibitor, stem-cell division inhibitors, stipiamide, streptonigrin, streptozocin, stromelysin inhibitors, sulfinosine, sulofenur, superactive vasoactive intestinal peptide antagonist, suradista, suramin, swainsonine, synthetic glycosaminoglycans, talisomycin, tallimustine, tamoxifen methiodide, tauromustine, tazarotene, tecogalan sodium, tegafur, tellurapyrylium, telomerase inhibitors, teloxantrone hydrochloride, temoporfin, temozolomide, teniposide, teroxirone, testolactone, tetrachlorodecaoxide, tetrazomine, thaliblastine, thalidomide, thiamiprine, thiocoraline, thioguanine, thiotepa, thrombopoietin, thrombopoietin mimetic, thymalfasin, thymopoietin receptor agonist, thymotrinan, thyroid stimulating hormone, tiazofurin, tin ethyl etiopurpurin, tirapazamine, titanocene dichloride, topotecan hydrochloride, topsentin, toremifene, toremifene citrate, totipotent stem cell factor, translation inhibitors, trestolone acetate, tretinoin, triacetyluridine, triciribine, triciribine phosphate, trimetrexate, trimetrexate glucuronate, triptorelin, tropisetron, tubulozole hydrochloride, turosteride, tyrosine kinase inhibitors, tyrphostins, UBC inhibitors, ubenimex, uracil mustard, uredepa, urogenital sinus-derived growth inhibitory factor, urokinase receptor antagonists, vapreotide, variolin B, velaresol, veramine, verdins, verteporfin, vinblastine sulfate, vincristine sulfate, vindesine, vindesine sulfate, vinepidine sulfate, vinglycinate sulfate, vinleurosine sulfate, vinorelbine, vinorelbine tartrate, vinrosidine sulfate, vinxaltine, vinzolidine sulfate, vitaxin, vorozole, zanoterone, zeniplatin, zilascorb, zinostatin, zinostatin stimalamer, or zorubicin hydrochloride.

### In Vivo Pharmacokinetics Properties of the Liposomal Elinafide Compositions

The liposomal composition disclosed herein may be formulated for oral, intravenous, intramuscular, intraperitoneal, or rectal delivery. Bioavailability is often assessed by comparing standard pharmacokinetic (PK) parameters such as Cₘₐₓ and AUC.

In one embodiment, the liposomal composition may produce a plasma PK profile characterized by elinafide plasma levels greater than free elinafide. In another embodiment, the Cₘₐₓ may be 5-fold, 10-fold, 20-fold, 30-fold, 40-fold, 50-fold, 60-fold, 70-fold, 80-fold, 90-fold, 100-fold, 150-fold, 200-fold, 250-fold, 300-fold, 350-fold, 400-fold, 450-fold, 500-fold, 550-fold, 600-fold, 650-fold, 700-fold, 750-fold, 800-fold, 850-fold, 900-fold, 950-fold, or 1000-fold greater than free elinafide.

In one embodiment, the liposomal composition may produce a plasma PK profile characterized by elinafide tumor levels greater than free elinafide In another embodiment, the elinafide tumor level may be 5-fold, 10-fold, 15-fold, 20-fold, 25-fold, 30-fold, 35-fold, 40-fold, 45-fold or 50-fold greater than free elinafide.

In an additional embodiment, the liposomal composition may produce a plasma PK profile characterized by AUC for elinafide from about 1,000 hr·ng/ml to about 55,000 hr·ng/ml, from about 5,000 hr·ng/ml to about 45,000 hr·ng/ml, from about 10,000 hr·ng/ml to about 35,000 hr·ng/ml, or from about 15,000 hr·ng/ml to about 25,000 hr·ng/ml. In another embodiment, the AUC_{inf} for elinafide may be about 1,000, 5,000, 10,000, 15,000, 20,000, 25,000, 30,000, 35,000, 40,000, 45,000, or 50,000 hr·ng/ml.

In an additional embodiment, the liposomal composition may produce a plasma PK profile characterized by t_{1/2} for elinafide from about 1 hour to about 25 hours, from about 5 hours to about 20 hours, from about 5 hours to about 15 hours, or from about 5 hours to about 10 hours. In another embodiment, the t_{1/2} for elinafide from may be about 1, 2, 3, 4, 5, 6, 7, 8, 9, 10, 11, 12, 13, 14, 15, 16, 17, 18, 19, 20, 21, 22, 23, 24, or 25 hours.

In an additional embodiment, the liposomal composition may produce a plasma PK profile characterized by clearance (CL) for elinafide below about 10,000 ml/hr/kg, about 9,000 ml/hr/kg, about 8,000 ml/hr/kg, about 7,000 ml/hr/kg, about 6,000 ml/hr/kg, about 5000 ml/hr/kg, about 4,000 ml/hr/kg, 3000 ml/hr/kg, about 2900 ml/hr/kg, about 2800 ml/hr/kg, about 2700 ml/hr/kg, about 2600 ml/hr/kg, about 2500 ml/hr/kg, about 2400 ml/hr/kg, about 2300 ml/hr/kg, about 2200 ml/hr/kg, about 2100 ml/hr/kg, about 2000 ml/hr/kg, about 1900 ml/hr/kg, about 1800 ml/hr/kg, about 1700 ml/hr/kg, about 1600 ml/hr/kg, about 1500 ml/hr/kg, about 1400 ml/hr/kg, about 1300 ml/hr/kg, about 1200 ml/hr/kg, about 1100 ml/hr/kg, about 1000 ml/hr/kg, about 900 ml/hr/kg, about 800 ml/hr/kg, about 700 ml/hr/kg, 600 ml/hr/kg, about 500 ml/hr/kg, about 400 ml/hr/kg, 300 ml/hr/kg, about 200 ml/hr/kg, or about 100 ml/hr/kg.

In an additional embodiment, the liposomal composition may produce a plasma PK profile characterized by steady state volume of distribution (Vss) for elinafide below about 100,000 ml, about 90,000 ml, about 80,000 ml, about 70,000 ml, about 60,000 ml, about 50,000 ml, about 40,000 ml, about 30,000 ml, about 20,000 ml, 10,000 ml, about 9,000 ml, about 8,000 ml, about 7,000 ml, about 6,000 ml, about 5,000 ml, about 4,000 ml, about 3,000 ml, about 2,000 ml, about 1,500 ml, or about 1,000 ml.

### IV. Examples

### Example 1. Preparation of Liposomal Elinafide

Elinafide was synthesized as described below:

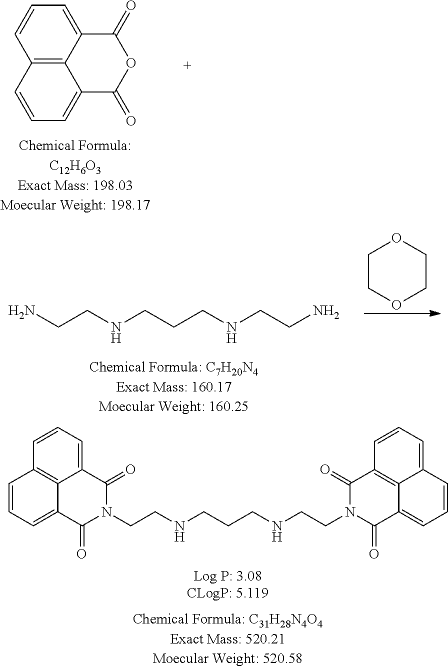 [SMILES: O=C1OC(=O)c2cccc3cccc1c23 ; C1COCCO1 ; NCCNCCCNCCN ; O=C1c2cccc3cccc(c23)C(=O)N1CCNCCCNCCN1C(=O)c2cccc3cccc(c23)C1=O]

Into a 250 mL round bottomed flask was charged 1.6 g bis-aminoethylpropanediamine and 40 mL 1,4-dioxane. To this solution was added 4 g 1,8-naphthalenedicarboxylic acid anhydride. The resulting mixture was heated to 105-110° C. (reflux of 1,4-dioxane at 101° C.) for 5 h under nitrogen, then allowed to cool to room temperature (RT) overnight. The resulting amber liquid with some dark precipitate was filtered through a Whatman filter and the dark precipitate was washed with dioxane. The dioxane layer was shown to have the desired product by HPLC/MS. The dioxane solution (amber colored) was placed in the refrigerator and overnight the solution solidified (dioxane melting point 12° C.). Upon thawing to RT, a beige solid resulted. This solid was collected by filtration and air dried. The recovered solid was dissolved in ca. 60 mL hot toluene (70° C.). The hot toluene solution was filtered through a Whatman filter and the toluene filtrate allowed to cool to RT and then at 4° C. overnight.

### Preparation of Liposome

Liposomes were prepared as follows. DSPC, cholesterol, and DSPE-PEG(2000) (55:40:5 percent molar ratio) were weighed into a 1 L round bottom flask and dissolved in 10 mL ethanol at 70° C. A 250 mM ammonium citrate solution (5.66 g of ammonium citrate in 100 mL of DI water, pH 5.01) or 250 mM ammonium sulfate was rapidly added to the lipid-ethanol solution at 70° C. resulting in the formation of crude vesicles, and heated at 70° C. for 30 minutes.

The crude vesicles were extruded through double stacked 200 nm membranes at 70° C. to produce a uniform particle size of approximately 200 nm (nitrogen used for pressure, 5 times @ ca. 200 psi). The resulting liposome mixture is then extruded through double stacked 100 nm membranes at 70° C. (nitrogen used for pressure 5-10 times @ ca. 400 psi). The particle size of the liposome mixture were measured using light scattering Malvern Zetasizer (100 μl of liposome mixture diluted with 900 μl of 0.9% saline): volume mean=93.3 d. nm; 90% volume diameter=132 d. nm; 10% volume diameter=62.9 d. nm; PdI=0.041; and pH=5.09.

To remove unencapsulated ammonium citrate, the liposomal mixture was diafiltered against a 300 mM sucrose solution (filtered through 0.22 micron filter membrane) using a Spectrum Laboratory Inc. Kros system unit with a Spectrum Filter Module, mPES/500 KD, surface area 790 cm². Particle size was measured on a Malvern Zetasizer using 100 uL of sample diluted with 900 uL of saline: volume mean=94.6 d. nm (final); 90% volume diameter=137 d. nm; 10% volume diameter=62.3 d. nm; PdI=0.055; Zeta potential=−1.00 mV (Dip cell used); and pH=6.37.

Remote Loading of Elinafide into Liposomes

Elinafide was weighed out (288.5 mg) into a 100 mL serum vial with 70 mL of 0.3 M sucrose solution (filtered through 0.22 micron filter membrane). The resulting slurry was dissolved using 1 N HCl (approximately 1.1 mL) with stirring for about 1 h (pH 4.9). The solution was brought to cloud point by adding 0.1 N NaOH (approximately 0.2 mL) (pH 5.4). The solution was filtered through a 0.2 sM syringe filter. The concentration of elinafide solution was 4.05 mg/mL.

20 mL of liposome (DSPC:Chol:DSPE-PEG2000, 55:40:5) was pipetted into a 60 mL serum vial. 20 mL of the 4.05 mg/mL elinafide solution was pipetted into a second 60 mL serum vial. Both the liposome and elinafide vials were heated to 60° C. with stirring for 15 min. At 60° C., the elinafide solution was rapidly added to the liposome with stirring. Heating was continued for 20 minutes. The mixture was allowed to cool to RT and stored at 4° C. until purification.

Unencapsulated elinafide was removed by diafiltration against 20 mM histine saline buffer (12.42 g of histidine in 4 L of saline adjusted to pH 6.8 using 1 N aqueous HCl) using a Spectrum Laboratory Inc. Kros system unit with a Spectrum Filter Module P/N: P-DI-500E-100-01N, mPE 5/500 KD, surface area 115 cm2. After diafiltration with 10 volumes of buffer (400 mL), the material was ultrafiltered to ca. 20 mL. Particle size was measured on a Malvern Zetasizer using 10 uL of sample diluted with 90 uL of 0.9% saline.

The method described above was used to prepare the liposomal elinafide formulations summarized in Table 1. Additional liposomal elinafide formulations were prepared and summarized in Table 9.

TABLE 1
Liposomal Elinafide Formulations

| | | | | Total | | |
|---|---|---|---|---|---|---|
| | | Loading | Particle | Lipids | Total Drug | Lipid/Drug |
| Example | Liposome | Agent | Size | (mg/mL) | (mg/mL) | Ratio |
| 1a | DSPC/Chol/DSPE- | Ammonium | 97.8 | 13.89 | 3.2 | 4.3 |
| | PEG(2000)(55/40/5) | Citrate | | | | |
| 1b | DSPC/Chol/DSPE- | Ammonium | 104.2 | 17.69 | 2.0 | 8.8 |
| | PEG(2000) (55/40/5) | Citrate | | | | |
| 1c | DSPC/Chol/DSPE- | Ammonium | 106.3 | 17.67 | 1.0 | 17.7 |
| | PEG(2000) (55/40/5) | Citrate | | | | |
| 1d | DSPC/Chol/DSPE- | Ammonium | 95.7 | 20.70 | 4.5 | 4.6 |
| | PEG(2000) (55/40/5) | Sulfate | | | | |
| 1e | DSPC/Chol/DSPE- | Ammonium | 103.5 | 17.96 | 2.1 | 8.6 |
| | PEG(2000) (55/40/5) | Sulfate | | | | |
| 1f | DSPC/Chol/DSPE- | Ammonium | 102.0 | 19.54 | 1.1 | 17.8 |
| | PEG(2000) (55/40/5) | Sulfate | | | | |

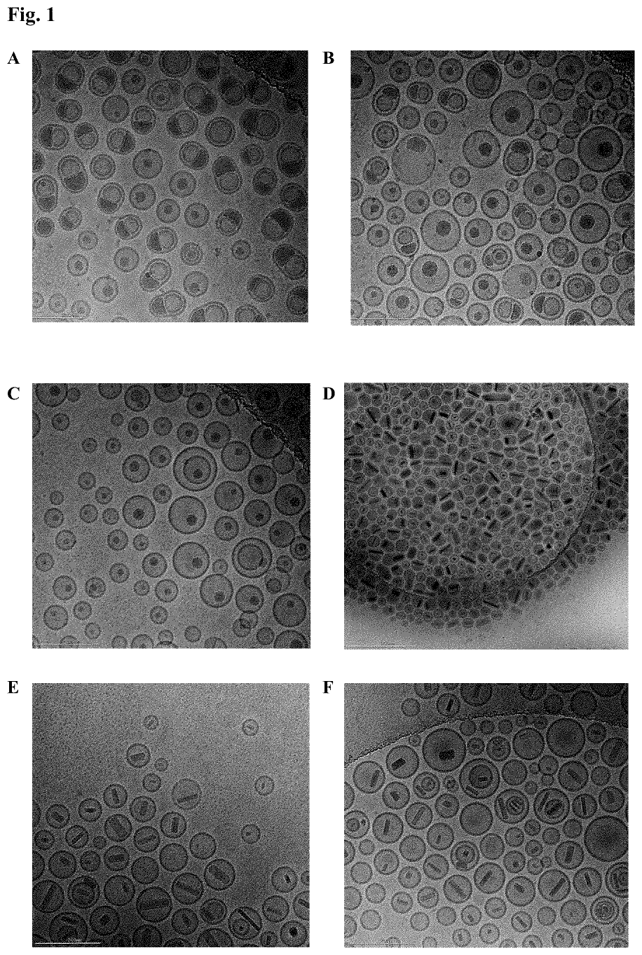

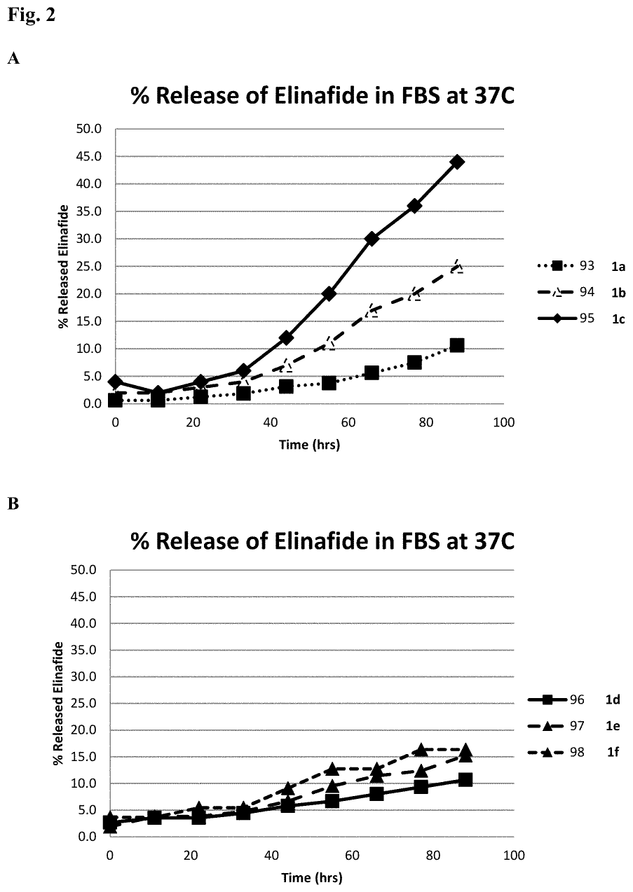

The elinafide liposomes were studies under transmission electron cryomicroscopy (cryo-TEM) as shown in FIG. 1. The topographical distribution of elinafide remotely loaded into liposomes containing ammonium citrate or ammonium sulfate counterparts are distinctly different. In ammonium citrate encapsulated liposomes, the cryo-TEM show dense dark centers or dark dense regions which appear to be full vesicles with a second vesicle entrapped within the larger structure. On the other hand, ammonium sulfate encapsulated liposomes produce rod like structures within the aqueous interior of the liposome. The highest amount of elinafide loaded (as based on a lipid to elinafide ratio), example 1f, gave images that indicate distortion of the vesicles, perhaps due to overloading with elinafide.

In Vitro Release of Elinafide from Liposomes

The release of elinafide from liposomes was determined for examples 1a-1f (Table 1) using an in vitro release assay in fetal bovine serum (FBS) at 37° C. A HPLC two-dimensional assay was used to separate free elinafide from liposomal elinafide followed by quantitative determination of free elinafide. The assay provides biological in vitro release data, i.e., the presence of relevant protein concentrations, and mimics in vivo conditions.

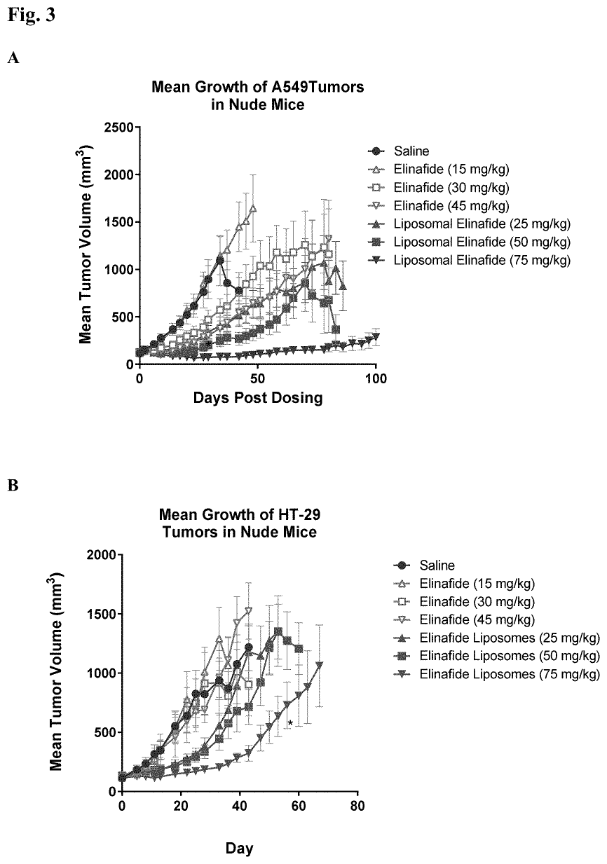

Elinafide release from liposomes as a function of time is shown in FIG. 2. The release of elinafide from liposomes is significantly delayed, i.e., less than 5% over 20 hrs and approximately 5% over 40 hr. Such slow release is thought to contribute to the reduction in myelosuppression, neuromuscular toxicity, and QTc prolongation attributed to elinafide.

FIG. 2 also shows that liposomes containing ammonium sulfate significantly delayed in vitro release of elinafide compared to liposomes containing ammonium citrate. Without wishing to be bound by any particular theory, it is believed that the in vitro release reflects differences in the morphology of the entrapped elinafide salt.

### Determination of In Vitro ICS of Liposomal Elinafide

HT-29 human colorectal adenocarcinoma cells (#HTB-38, ATCC, Manassas, Va.) were plated in 96-well tissue culture plates (Costar #3595) at 5×10³ cells/well in a final volume of 0.1 mL of 10% fetal bovine serum in McCoy's 5A (#10-050-CV, Mediatech, Manassas, Va.). Defined fetal bovine serum was obtained from HyClone (#SH30070.03, lot #AWB96395, Logan, Utah). Plates containing cells were incubated at 37° C. in 5% CO₂ in humidified air for 24 hr. The selected initial cell plating density was chosen based upon the approximate doubling time of the human tumor cell line.

Test compounds were diluted from stock solutions to 2.2 mmol/L in Dulbecco's modified phosphate-buffered saline (DPBS; Mediatech, Inc., lot #21031339, Manassas, Va.), then serially diluted three-fold in DPBS to generate a nine point dose-response curve. Ten microliters of diluted test compounds were added to wells in triplicate to achieve the desired final concentration of test compounds. Plates containing cells with and without added test compounds were returned to incubation as described above.

For the two hour cytotoxicity assessment, medium was removed after two hours of drug exposure and replaced with 0.1 mL/well culture medium, and cells were incubated for an additional 70 hours as above.

For the twenty-four hour cytotoxicity assessment, medium was removed after one day of drug exposure and replaced with 0.1 mL/well culture medium, and cells were returned to incubation for an additional 48 hours. Subsequently, cell viability was assessed using Alamar Blue. For this purpose, media was removed by pipetting from cultured cells and replaced with 0.1 mL/well of 10% (v/v) Alamar Blue (#BUFO12A, AbD Serotec, Raleigh, N.C.) diluted in the appropriate cell culture media. Plates were then returned to incubation as before for appropriate color development, between two to four hours.

Fluorescence of individual plate wells was measured at 545 nm/590 nm (excitation/emission) using a BioTek Synergy4 microplate reader. Cell viability was calculated as a percentage of measured fluorescence obtained relative to cells treated with culture media alone. IC₅₀ values (μmol/L) were determined with the mean of triplicate values using nonlinear regression analysis and a four-parameter logistic model.

Table 2 reports the IC₅₀ values of examples 1a-1f at 2 hrs and 24 hrs post-treatment.

TABLE 2
IC₅₀ values for Liposomal Elinafide Formulations

| | | | 2 hr Tx IC50 | 24 hr Tx IC50 |
|---|---|---|---|---|
| Examples | Liposome | Salt | [μM] | [μM] |
| 1a | DSPC/Chol/DSPE- | Ammonium | >20 | 0.040 |
| | PEG(2000) (55/40/5) | citrate | | |
| 1b | DSPC/Chol/DSPE- | Ammonium | 0.654 | 0.039 |
| | PEG(2000) (55/40/5) | citrate | | |
| 1c | DSPC/Chol/DSPE- | Ammonium | >20 | 0.047 |
| | PEG(2000) (55/40/5) | citrate | | |
| 1d | DSPC/Chol/DSPE- | Ammonium | 1.623 | 0.181 |
| | PEG(2000) (55/40/5) | sulfate | | |
| 1e | DSPC/Chol/DSPE- | Ammonium | 1.244 | 0.474 |
| | PEG(2000) (55/40/5) | sulfate | | |
| 1f | DSPC/Chol/DSPE- | Ammonium | >20 | 0.026 |
| | PEG(2000) (55/40/5) | sulfate | | |

The IC₅₀ values obtained from liposomal elinafide demonstrate that delayed release of elinafide results in improved efficacy over time. For instance, IC₅₀ values were significantly lower after 24 hr treatment than at 2 hrs.

### Example 2. In Vivo Anti-Tumor Activity of Liposomal Elinafide

Antitumor activity of liposomal elinafide on the growth of established human tumor cell line xenografts in athymic nude mice was studied to determined whether liposomal elinafide could provide greater efficacy than free elinafide at equivalent maximum tolerated dose (MTD).

Tumor cell lines were implanted subcutaneously into the flank of athymic nude mice and allowed to grow to a fixed size. Mice that did not grow tumors were rejected. Mice were allocated to receive either saline (control, included in all studies), free elinafide, or liposomal elinafide, and administered the designated treatment by slow bolus intravenous injection. In each case, where possible, doses were selected as providing equivalent levels of toxicity/tolerance. Tumor volume was analyzed to determine tumor growth delay (TGD) as defined as the difference in days for the median tumor volume of the treatment group and the saline treated group to reach either 500 mm³ or 1000 mm³ in size, depending on the tumor model. Mice were removed from the study if they lost 20% of their initial bodyweight or became moribund or if their tumor volume exceeded 2500 mm³ or the tumor ulcerated. If less than half of the initial cohort of mice remained, that group was no longer graphed or included in further tumor analysis. However, any remaining animals were followed until completion of the in-life observation period and included in a survival analysis. The variable features of this study are summarized in Table 3.

TABLE 3
Summary of In Vivo Antitumor Activity
Studies in Athymic Nude Mice

Doses (mg/kg)

| | | | | Liposomal |
|---|---|---|---|---|
| Tumor | Cell Line | No./group | Elinafide | Elinafide |
| NSCLC | A549 | 10 | 15, 30, 45 | 25, 50, 75 |
| Colorectal | HT29 | 10 | 15, 30, 45 | 25, 50, 75 |
| Panreatic | BxPC3 | 10 | 15, 30, 45 | 25, 50, 75 |

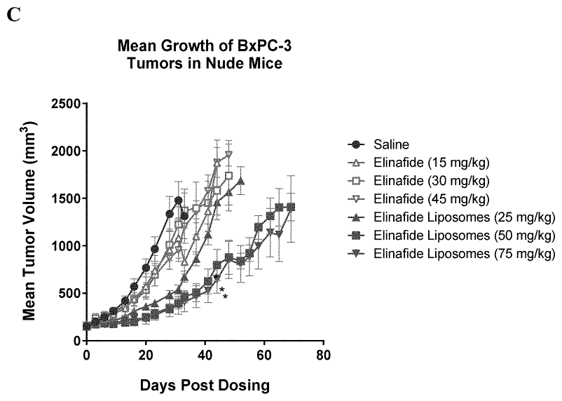

The study demonstrate that liposomal elinafide acts as an active antitumor agent in these xenograft models, and possesses significantly greater antitumor activity compared to comparably tolerated doses of free elinafide. The effects of liposomal elinafide on tumor volume are illustrated in FIG. 3. A single dose of liposomal elinafide increase TGD by 26, 44, and >200 days relative to saline treated mice.

In mice bearing A549 tumor cells, liposomal elinafide (50 and 75 mg/kg) increased TGD greater than all doses of free elinafide as shown in FIG. 3A. Likewise, in mice bearing HT29 tumors, liposomal elinafide increased TGD by 10, 14 and 33 days at doses of 25, 50 and 75 mg/kg compared to saline which was also greater than all doses of free elinafide as shown in FIG. 3B. In mice bearing BxPC3 tumor cells, liposomal elinafide similarly increased TGD compared to all doses of elinafide as shown in FIG. 3C.

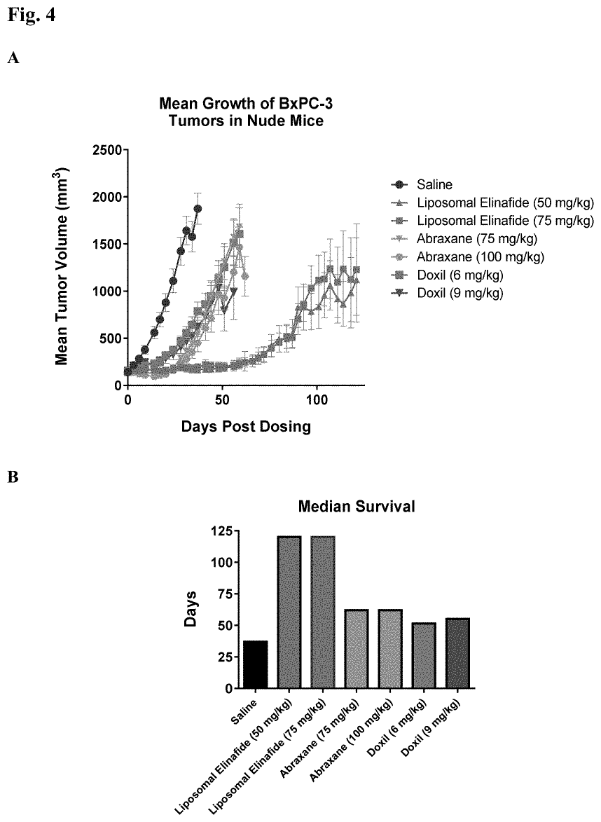

Liposomal elinafide (50 and 75 mg/kg) also proved superior to commercial formulations of Abraxane (nab-Paclitaxel, 75 and 100 mg/kg) and DOXIL (liposomal doxorubicin, 6 and 9 mg/kg) in delaying tumor growth or reducing tumor volume in mice bearing BxPC3 tumors. As shown in FIG. 4A, liposomal elinafide increased TGD compared to Abraxane and DOXIL. Moreover, liposomal elinafide significantly (p<0.05) increased the median survival of such mice compared to saline, Abraxane, and DOXIL as shown in FIG. 4B.

### Pharmacokinetic of Liposomal Elinafide

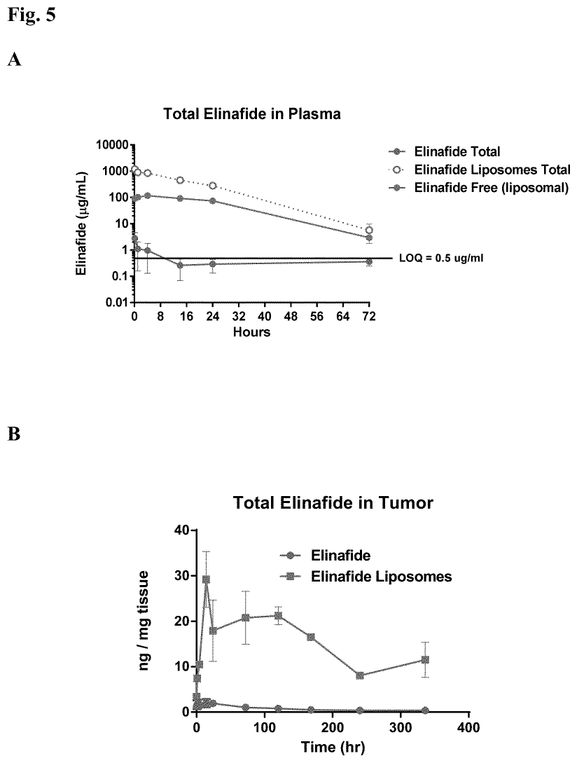

A biodistribution and pharmacokinetic study of liposomal elinafide in athymic nude mice bearing HT29 Human colorectal xenografts was also conducted. Treatment with liposomal elinafide (45 mg/kg oxaliplatin) increased total plasma elinafide exposure (AUC) at least 1000-fold greater than treatment with the same dose of free elinafide (FIG. 5A) and, at the same time, decrease drug clearance (Cl) and steady state volume of distribution (Vss). Interestingly, the total tumor elinafide exposure (AUC) was 5- to 30-fold greater than treatment with the same dose of free elinafide (FIG. 5B). Table 4 provides a summary of the liposomal elinafide and free elinafide pharmacokinetics in rats demonstrating similar increases in elinafide exposure (AUC) following administration of liposomal elinafide.

TABLE 4
Pharmacokinetics of Liposomal Elinafide and Free Elinafide

| | Cmax | AUC | Half-Life | Cl | Vss |
|---|---|---|---|---|---|
| Treatment (mg/kg) | (ng/mL) | (hr*ng/mL) | (hr) | (ml/hr/kg) | (mL) |
| Free Elinafide @ 20 | 1.4 | 4.7 | 53.5 | 3902563.7 | 203766537.5 |
| Liposomal Elinafide @ 30 | 125 | 23463.7 | 24.1 | 1294.1 | 44844.2 |

These observations are consistent with 1) EPR effect, and 2) sustained plasma levels of liposomal elinafide compare to free elinafide within the circulation and tumor microenvironment. Without wishing to be bound by any particular theory, it is believed that the increased tumor exposure to elinafide likely contributed to the increased efficacy observed with liposomal elinafide in the tumor xenograft models discussed above.

### Effect of Liposomal Elinafide on Muscle Myopathy

In a single-dose study, rats were treated with liposomal elinafide (50 and 60 mg/kg) or free elinafide (25 and 35 mg/kg) at MTD and observed for a 14-day period. Rats that loss greater than 20% of its body weight were dropped from the study. After 14 days, the hind leg muscle of each treated rat was examined for necrosis, inflammation, and/or degeneration. The results of the study are present in Table 5 below.

TABLE 5
Effect of Liposomal Elinafide on Muscle Myopathy

| | Dose | | | |
|---|---|---|---|---|
| Treatment | (mg/kg) | Sex | Survival | Myopathy |
| Free | 25 | M | 3/3 | 2/3 |
| Elinafide | 25 | F | 3/3 | 1/3 |
| | 35 | M | 0/3 | — |
| | 35 | F | 3/3 | 3/3 |
| Liposomal | 50 | M | 3/3 | 0/3 |
| Elinafide | 50 | F | 3/3 | 0/3 |
| | 60 | M | 1/3 | 0/1 |
| | 60 | F | 3/3 | 0/3 |

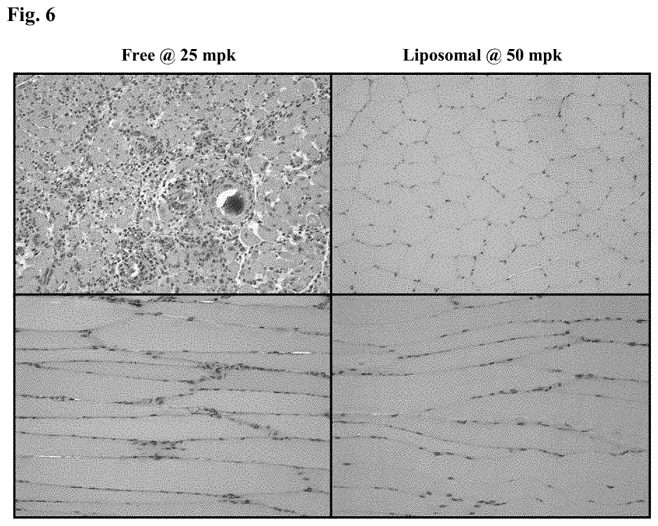

Administration of liposomal elinafide at MTD did not induce muscle myopathy in any of the treated rats. In contrast, rats treated with free elinafide experienced significant muscle myopathy. At the highest MTD of free elinafide, all rats showed symptoms of muscle myopathy. FIG. 6 provides cross (top) and longitudinal (bottom) sections of the quadriceps femoris of male rats treated with either free elinafide (25 mg/kg) or liposomal elinafide (50 mg/kg).

This study demonstrated that liposomal elinafide has reduced side effects, such as muscle myopathy. It is further expected that liposomal elinafide would also reduce myelosuppression, neuromuscular toxicity, and QTc prolongation.

### Example 3. In Vitro IC₅₀ Determination of Other Liposomal Naphthalimides and Azonifides

Liposomal formulations of other naphthalimides and azonifides were also prepared. Table 6 provides a summary of the naphthalimides and azonifides liposomal formulations.

TABLE 6
Liposomal Naphthalimide and Azonafide Formulations

| | | Loading | Particle | Total Lipids | Total Drug | Lipid/Drug |
|---|---|---|---|---|---|---|
| Payload | Liposome | Agent | Size | (mg/mL) | (mg/mL) | Ratio |
| Amonafide | DSPC/Chol/DSPE- | Ammonium | 107.5 | 17.63 | 4.7 | 3.8 |
| | PEG(2000) (55/40/5) | Sulfate | | | | |
| Amonafide | DSPC/Chol/DSPE- | Ammonium | 106.7 | 17.78 | 4.4 | 4.0 |
| | PEG(2000) (55/40/5) | Citrate | | | | |
| Azonifide | DSPC:Chol:DSPE- | Ammonium | 102.4 | n.d.^{a} | 3.59 | n.d. |
| | PEG(2000) 55:40:5 | Sulfate | | | | |
| Azonifide | DSPC:Chol:DSPE- | Ammonium | 99.4 | n.d. | 4.22 | n.d. |
| | PEG(2000) 55:40:5 | Citrate | | | | |
| UNBS-5162 | DSPC:Chol:DSPE- | Ammonium | 103.6 | n.d. | 3.23 | n.d. |
| | PEG(2000) 55:40:5 | Sulfate | | | | |
| UNBS-5162 | DSPC:Chol:DSPE- | Ammonium | 99.5 | n.d. | 2.3 | n.d. |
| | PEG(2000) 55:40:5 | Citrate | | | | |
| Ethonafide | DSPC:Chol:DSPE- | Ammonium | 101.1 | n.d. | 3.06 | n.d. |
| | PEG(2000) 55:40:5 | Sulfate | | | | |
| Ethonafide | DSPC:Chol:DSPE- | Ammonium | 100.2 | n.d. | 1.37 | n.d. |
| | PEG(2000) 55:40:5 | Citrate | | | | |

^{a}n.d. = not determined

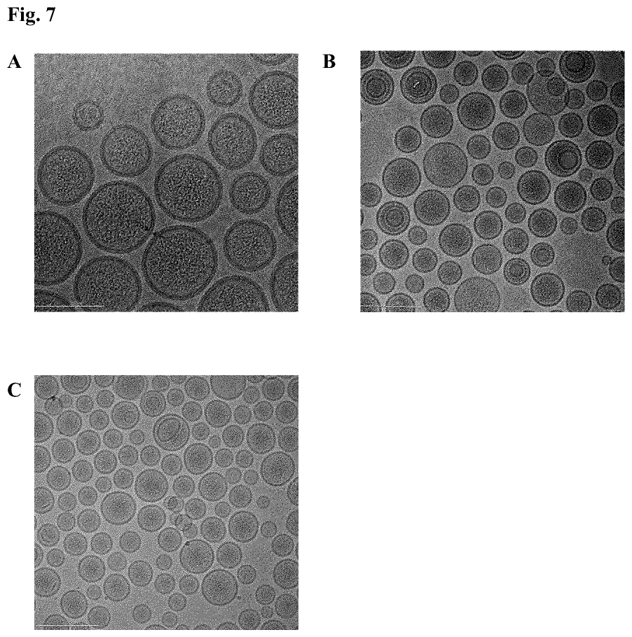

FIG. 7 show the cryo-TEM for liposomal amonifide and UNBS-5162 formulations.

In vitro IC₅₀ values were also determined for the liposomal naphthalimides and azonifides formulations using the same method as described above in Example I. IC₅₀ values are summarized in Table 7.

TABLE 7
Summary of IC₅₀ Determination For Naphthalimide and Azonafide Formulations

| | | | 2 hr Tx | 24 hr Tx | 48 hr Tx | 72 hr Tx |
|---|---|---|---|---|---|---|
| Payload | Liposome | Salt | (μM) | (μM) | (μM) | (μM) |
| Amonafide | Free | N/A | 90.3 | 34.2 | 15.7 | N/A |
| Amonafide | DSPC/Chol/DSPE- | Sulfate | 323.3 | 10.4 | 5.29 | N/A |
| | PEG(2000) (55/40/5) | | | | | |
| Amonafide | DSPC/Chol/DSPE- | Citrate | >500 | 9.06 | 5.08 | N/A |
| | PEG(2000) (55/40/5) | | | | | |
| Azonifide | Free | HCl | 1.170 | 0.256 | 0.283 | 0.386 |
| Azonifide | DSPC:Chol:DSPE- | Sulfate | 1.453 | 0.238 | 0.179 | 0.353 |
| | PEG(2000) 55:40:5 | | | | | |
| Azonifide | DSPC:Chol:DSPE- | Citrate | 1.163 | 0.304 | 0.142 | 0.312 |
| | PEG(2000) 55:40:5 | | | | | |
| UNBS-5162 | Free | HCl | >100 | 24.8 | N/A | 31.3 |
| UNBS-5162 | DSPC:Chol:DSPE- | Sulfate | >100 | >100 | N/A | >100 |
| | PEG(2000) 55:40:5 | | | | | |
| UNBS-5162 | DSPC:Chol:DSPE- | Citrate | >100 | >100 | N/A | >100 |
| | PEG(2000) 55:40:5 | | | | | |
| Ethonafide | Free | HC1 | 0.751 | 0.189 | N/A | 0.388 |
| Ethonafide | DSPC:Chol:DSPE- | Sulfate | 5.39 | 0.437 | N/A | 0.340 |
| | PEG(2000) 55:40:5 | | | | | |
| Ethonafide | DSPC:Chol:DSPE- | Citrate | 6.90 | 0.333 | N/A | 0.348 |
| | PEG(2000) 55:40:5 | | | | | |

As shown in Table 7, a higher IC₅₀ values at 2 hr vs. longer exposure time points for the liposomal formulations generally indicates extended release of drug (except for azonifide).

### Example 4. In Vivo Anti-Tumor Activity of Other Liposomal Naphthalimides and Azonifides

Antitumor activity of other liposomal naphthalimides and azonifides on the growth of HT29 human tumor cell line xenograft in male athymic nude mice was evaluated. The MTD of liposomal and free naphthalimides and azonifides are provided below in Table 8.

TABLE 8
MTD of liposomal and free naphthalimides and azonifides

| Treatment | MTD (mg/kg) |
|---|---|
| Amonafide free | 45 |
| Amonafide Citrate | 120* |
| Amonafide Sulfate | 120* |
| Azonafide free | 10 |
| Azonafide Citrate | 30 |
| Azonafide Sulfate | 30 |
| Ethonafide free | 15 |
| Ethonafide Citrate | 30 |
| Ethonafide Sulfate lipo | 30 |
| UNBS-5162 free | 45 |
| UNBS-5162 Citrate | 200* |
| UNBS-5162 Sulfate lipo | 200* |

*Volume limited

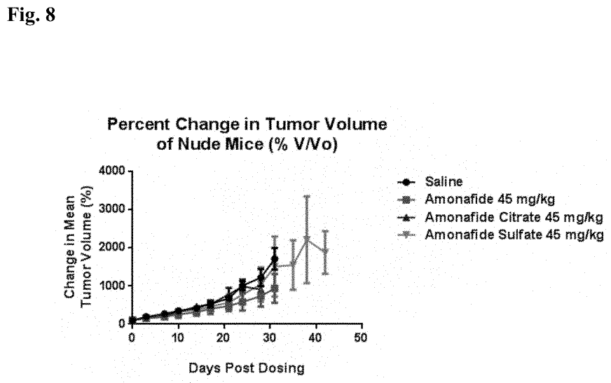

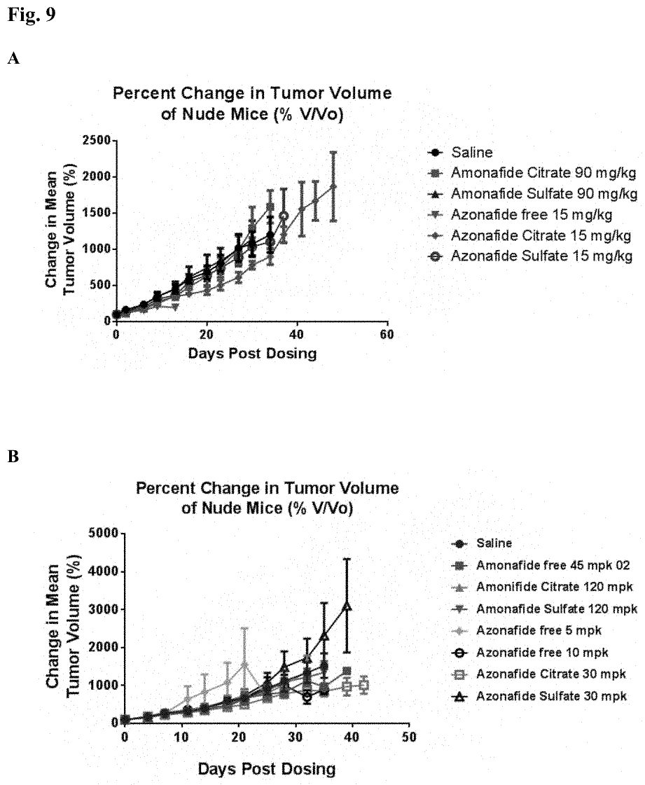

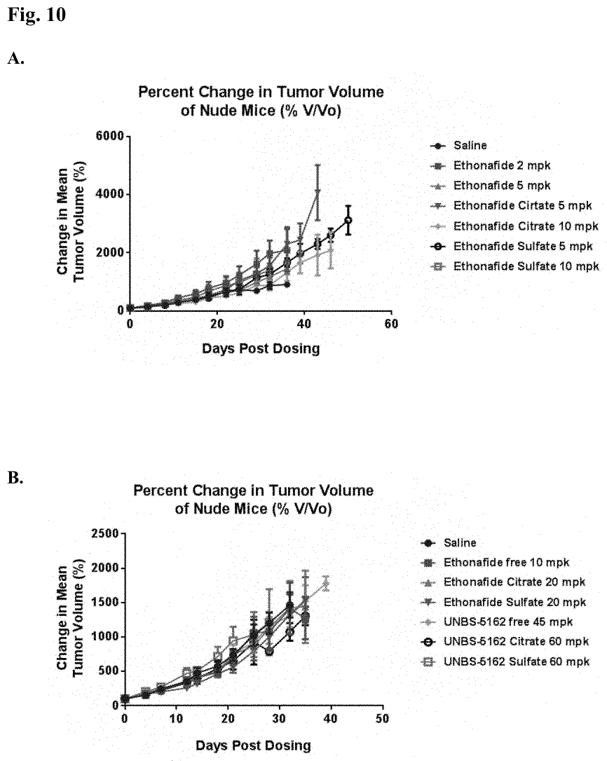

FIGS. 8 to 10 show the percent change in tumor volume following administration of liposomal and free naphthalimides and azonifides. Although liposomal formulations of amonafide, azonafide, UNBS-5162, and ethonafide were successfully prepared (using a process identical to elinafide), there was no significant gain in efficacy in a HT29 tumor xenograft model (single dose studies). In each instance, even though a greater amount of active drug could be dosed (greater MTD) in the liposomal formulations than for the free drugs, it did not have a significant impact on anti-tumor efficacy. Surprisingly, only elinafide demonstrated improved in vivo anti-tumor efficacy, i.e., significantly delayed tumor growth in three tumor xenograft models.

Table 9 provides a summary of additional liposomal elinafide formulations and their IC₅₀ values and provide for additional benefits under suitable conditions.

TABLE 9
Additional Liposomal Elinafide Formulations

| | | | IVR | | Total | Total | | 2 hr Tx | 24 hr Tx |
|---|---|---|---|---|---|---|---|---|---|
| | | | (60° C., | Particle | Lipid | Elinafide | | [μM] | [μM] |
| Sample ID | Liposome | Salt | 72 h) | Size | (mg/ml) | (mg/ml) | Lipid:Drug | (HT29) | (HT29) |
| E000389-146 | DSPC:Chol:DSPE- | sulfate | Slow | 99 | 21.8 | 3.32 | 6.6 | >30 | 3.54 |
| | PEG(2000) 55:40:5 | | | | | | | | |
| E000389-149 | DSPC:Chol:DSPE- | citrate | Moderate | 101.3 | 18 | 2.07 | 8.7 | >30 | 11.56 |
| | PEG(2000) 55:40:5 | | | | | | | | |
| E000389-150 | DSPC:Chol:DSPE- | sulfate | Slow | 100.2 | 21.4 | 3.57 | 6.0 | >30 | 0.89 |
| | PEG(2000) 55:40:5 | | | | | | | | |
| E000389-151 | DSPC:Chol:DSPE- | sulfate | Moderate | 102.8 | 17.1 | 2.43 | 7.0 | >30 | >30 |
| | PEG(2000) 55:40:5 | | | | | | | | |
| E000389-166 | DSPC:Chol:DSPE- | sulfate | Moderate | 100.6 | 17.8 | 1.92 | 9.3 | >30 | 3.97 |
| | PEG(2000) 55:40:5 | | | | | | | | |
| E000389-273 | DSPC:DSPG:Chol: | sulfate | ND | 98.5 | 21.2 | 3.70 | 5.7 | 0.387 | 0.307 |
| | DSPE-PEG(2000) | | | | | | | | |
| | 35:20:40:5 | | | | | | | | |
| E000389-274 | DSPC:DSPG:Chol: | sulfate | ND | 102.7 | 18 | 2.34 | 7.7 | 0.535 | 0.263 |
| | DSPE-PEG(2000) | | | | | | | | |
| | 35:20:40:5 | | | | | | | | |
| E000389-278 | DSPC:DSPG:Chol: | citrate | Moderate | 86.3 | 22.4 | 3.64 | 6.1 | 0.506 | 0.221 |
| | DSPE-PEG(2000) | | | | | | | | |
| | 35:20:40:5 | | | | | | | | |
| E000389-279 | DSPC:DSPG:Chol: | citrate | Moderate | 88.7 | 19.1 | 2.37 | 8.1 | 0.247 | 0.132 |
| | DSPE-PEG(2000) | | | | | | | | |
| | 35:20:40:5 | | | | | | | | |
| E000389-290 | DOPC:DOTAP:Chol: | sulfate | Very fast | 90.8 | 23.6 | 3.87 | 6.1 | 0.106 | 0.025 |
| | DSPE-PEG(2000) | | | | | | | | |
| | 35:30:30:5 | | | | | | | | |
| E000389-291 | DOPC:DOTAP:Chol: | sulfate | Very fast | 92.9 | 20.0 | 2.43 | 8.2 | 0.084 | 0.050 |
| | DSPE-PEG(2000) | | | | | | | | |
| | 35:30:30:5 | | | | | | | | |
| E000389-295 | DOPC:DOTAP:Chol: | sulfate | Very fast | 103.8 | 22.5 | 2.80 | 8.0 | 0.124 | 0.038 |
| | 35:20:45 | | | | | | | | |
| E000389-296 | DOPC:DOTAP:Chol: | sulfate | Very fast | 109.3 | 13.7 | 1.45 | 9.5 | 0.112 | 0.027 |
| | 35:20:45 | | | | | | | | |
| E000389-299 | DSPC:DSPG:Chol | sulfate | Moderate | 100.3 | 20.6 | 3.35 | 6.2 | 0.972 | 0.126 |
| | 35:20:45 | | | | | | | | |
| E000389-300 | DSPC:DSPG:Chol | sulfate | Moderate | 98.7 | 18.0 | 2.16 | 8.3 | 1.355 | 0.080 |
| | 35:20:45 | | | | | | | | |
| E000389-302 | DSPC:DSPE:Chol: | sulfate | Moderate | 92.1 | 23.7 | 3.64 | 6.5 | 0.440 | 0.151 |
| | DSPE-PEG(2000) | | | | | | | | |
| | 35:20:40:5 | | | | | | | | |
| E000389-303 | DSPC:DSPE:Chol: | sulfate | Moderate | 96.1 | 20.1 | 2.43 | 8.3 | 2.497 | 0.117 |
| | DSPE-PEG(2000) | | | | | | | | |
| | 35:20:40:5 | | | | | | | | |
| E000389-311 | DSPC:DSPE:Chol | sulfate | Moderate | 93.4 | 16.9 | 2.98 | 5.7 | 1.100 | 0.430 |
| | 35:20:45 | | | | | | | | |
| E000389-312 | DSPC:DSPE:Chol | sulfate | Moderate | 95.7 | 15.2 | 2.00 | 7.6 | 1.833 | 0.331 |
| | 35:20:45 | | | | | | | | |
| E000389-328 | DSPC:DSPG:Chol | citrate | Moderate | 114.4 | 22.8 | 3.19 | 7.1 | 0.903 | 0.144 |
| | 35:20:45 | | | | | | | | |
| E000389-329 | DSPC:DSPG:Chol | citrate | Moderate | 99.9 | 18.7 | 1.91 | 9.8 | 0.480 | 0.116 |
| | 35:20:45 | | | | | | | | |
| E000389-330 | DSPC:DSPE:Chol: | citrate | Moderate | 94.4 | 22.8 | 3.82 | 5.9 | 0.721 | 0.135 |
| | DSPE-PEG(2000) | | | | | | | | |
| | 35:20:40:5 | | | | | | | | |
| E000389-331 | DSPC:DSPE:Chol: | citrate | Moderate | 95 | 19.8 | 2.29 | 8.6 | 0.872 | 0.231 |
| | DSPE-PEG(2000) | | | | | | | | |
| | 35:20:40:5 | | | | | | | | |
| E000389-332 | DSPC:DSPE:Chol | citrate | Slow | 85.5 | 12.4 | 2.16 | 5.8 | 1.800 | 0.620 |
| | 35:20:45 | | | | | | | | |
| E000389-333 | DSPC:DSPE:Chol | citrate | Slow | 97.1 | 11.9 | 1.30 | 9.2 | 1.093 | 1.220 |
| | 35:20:45 | | | | | | | | |
| E000389-337 | DOPC:DOTAP:Chol | citrate | Very fast | 95.9 | 21.1 | 3.12 | 6.8 | 0.143 | 0.065 |
| | 35:20:45 | | | | | | | | |
| E000389-338 | DOPC:DOTAP:Chol | citrate | Very fast | 91.7 | 16.6 | 1.90 | 8.7 | 0.089 | 0.071 |
| | 35:20:45 | | | | | | | | |
| E000389-133 | DSPC:Chol:DSPE- | citrate | Slow | 96.4 | 19.3 | 4.17 | 4.6 | >30 | <0.0046 |
| | PEG(2000) 50:45:5 | | | | | | | | |
| E000389-153 | DSPC:Chol:DSPE- | citrate | Slow | 97.1 | 16.8 | 2.26 | 7.4 | >30 | <0.0046 |
| | PEG(2000) 50:45:5 | | | | | | | | |
| E000389-206 | DSPC:Chol:DSPE- | sulfate | ND | 97.2 | 20.06 | 3.78 | 5.3 | >20 | >20 |
| | PEG(2000) 50:45:5 | | | | | | | | |
| E000389-207 | DSPC:Chol:DSPE- | sulfate | Moderate | 100.6 | 16.29 | 2.27 | 7.2 | >20 | >20 |
| | PEG(2000) 50:45:5 | | | | | | | | |
| E000389-149 | DSPC:Chol:DSPE- | citrate | Moderate | 101.3 | 18 | 2.07 | 8.7 | >30 | 11.56 |
| | PEG(2000) 55:40:5 | | | | | | | | |
| E000389-146 | DSPC:Chol:DSPE- | sulfate | Slow | 99 | 21.8 | 3.32 | 6.6 | >30 | 3.54 |
| | PEG(2000) 55:40:5 | | | | | | | | |
| E000389-150 | DSPC:Chol:DSPE- | sulfate | Slow | 100.2 | 21.4 | 3.57 | 6.0 | >30 | 0.89 |
| | PEG(2000) 55:40:5 | | | | | | | | |
| E000389-151 | DSPC:Chol:DSPE- | sulfate | Moderate | 102.8 | 17.1 | 2.43 | 7.0 | >30 | >30 |
| | PEG(2000) 55:40:5 | | | | | | | | |
| E000389-166 | DSPC:Chol:DSPE- | sulfate | Moderate | 100.6 | 17.8 | 1.92 | 9.3 | >30 | 3.97 |
| | PEG(2000) 55:40:5 | | | | | | | | |
| E000389-152 | DSPC:Chol:DSPE- | citrate | Slow | 92.4 | 19.9 | 3.87 | 5.1 | >30 | 3.53 |
| | PEG(2000) 60:35:5 | | | | | | | | |
| E000398-188 | DSPC:Chol:DSPE- | citrate | Moderate | 94.7 | 19.7 | 2.48 | 8.0 | >20 | >20 |
| | PEG(2000) 60:35:5 | | | | | | | | |
| E000389-147 | DSPC:Chol:DSPE- | sulfate | Moderate | 98.5 | 21.1 | 3.05 | 6.9 | 0.57 | 0.15 |
| | PEG(2000) 60:35:5 | | | | | | | | |
| E000389-167 | DSPC:Chol:DSPE- | sulfate | Moderate | 100.4 | 17.6 | 2.24 | 7.9 | >30 | 0.63 |
| | PEG(2000) 60:35:5 | | | | | | | | |
| E000389-217 | DSPC:Chol:DSPE- | citrate | Moderate | 88.4 | 21.51 | 2.54 | 8.5 | 0.205 | 0.238 |
| | PEG(2000) 65:30:5 | | | | | | | | |
| E000389-213 | DSPC:Chol:DSPE- | sulfate | Moderate | 93.5 | 19.73 | 2.59 | 7.6 | 3.731 | 0.384 |
| | PEG(2000) 65:30:5 | | | | | | | | |
| E000389-154 | DSPC:Chol:DSPE- | citrate | Moderate | 92.4 | 15.8 | 1.87 | 8.5 | 11.1 | 0.665 |
| | PEG(2000) 70:25:5 | | | | | | | | |
| E000389-168 | DSPC:Chol:DSPE- | sulfate | Very fast | 90.2 | 15.3 | 0.83 | 18.4 | 0.61 | 0.06 |
| | PEG(2000) 70:25:5 | | | | | | | | |
| E000389-131 | DSPC:Chol:DSPE- | citrate | Fast | 87 | 17.7 | 1.59 | 11.1 | 1.73 | 0.16 |
| | PEG(2000) 80:15:5 | | | | | | | | |
| E000389-155 | DSPC:Chol:DSPE- | citrate | Fast | 85.8 | 16.6 | 1.32 | 12.6 | 15.9 | 1.187 |
| | PEG(2000) 80:15:5 | | | | | | | | |
| E000389-161 | DSPC:Chol:DSPE- | sulfate | Fast | 88.6 | 18.5 | 1.86 | 9.9 | 0.90 | 0.17 |
| | PEG(2000) 80:15:5 | | | | | | | | |
| E000389-162 | DSPC:Chol:DSPE- | sulfate | Fast | 92.3 | 15.9 | 1.76 | 9.0 | 0.94 | 0.14 |
| | PEG(2000) 80:15:5 | | | | | | | | |
| E000389-216 | DSPC:Chol:DSPE- | citrate | ND | 87.9 | 24.07 | 3.14 | 7.7 | 0.177 | 0.251 |
| | PEG(2000)65:30:5 | | | | | | | | |
| E000389-212 | DSPC:Chol:DSPE- | sulfate | Moderate | 89.9 | 22.64 | 2.74 | 8.3 | 3.645 | 0.512 |
| | PEG(2000)65:30:5 | | | | | | | | |
| E000389-134 | DSPC:Chol:DSPE- | citrate | Fast | 84.9 | 18.6 | 1.51 | 12.3 | 0.74 | 0.07 |
| | PEG(2000)70:25:5 | | | | | | | | |
| E000389-148 | DSPC:Chol:DSPE- | sulfate | Very fast | 89.1 | 18.5 | 0.85 | 21.8 | 0.27 | 0.07 |
| | PEG(2000)70:25:5 | | | | | | | | |
| E000389-146 | DSPC:Chol:DSPE- | sulfate | slow | 99 | 21.8 | 3.32 | 6.6 | >30 | 3.54 |
| | PEG(2000) 55:40:5 | | | | | | | | |
| E000389-149 | DSPC:Chol:DSPE- | citrate | moderate | 101.3 | 18 | 2.07 | 8.7 | >30 | 11.56 |
| | PEG(2000) 55:40:5 | | | | | | | | |
| E000389-150 | DSPC:Chol:DSPE- | sulfate | slow | 100.2 | 21.4 | 3.57 | 6.0 | >30 | 0.89 |
| | PEG(2000) 55:40:5 | | | | | | | | |
| E000389-151 | DSPC:Chol:DSPE- | sulfate | moderate | 102.8 | 17.1 | 2.43 | 7.0 | >30 | >30 |
| | PEG(2000) 55:40:5 | | | | | | | | |
| E000389-166 | DSPC:Chol:DSPE- | sulfate | moderate | 100.6 | 17.8 | 1.92 | 9.3 | >30 | 3.97 |
| | PEG(2000) 55:40:5 | | | | | | | | |
| E000398-195 | DSPC:Chol: 55:45 | citrate | slow | 97.3 | 23.3 | 3.63 | 6.4 | >20 | >20 |
| E000398-196 | DSPC:Chol: 55:45 | citrate | moderate | 97.3 | 20.4 | 2.41 | 8.5 | >20 | >20 |
| E000389-199 | DSPC:Chol: 55:45 | sulfate | ND | 99.7 | 18.8 | 3.59 | 5.2 | >20 | >20 |
| E000389-200 | DSPC:Chol:DSPE- | citrate | Very fast | 58.5 | 24.8 | 1.54 | 16.2 | 3.251 | 0.424 |
| | PEG(2000) 55:40:5 | | | | | | | | |
| E000389-201 | DSPC:Chol: 55:45 | sulfate | moderate | 106.3 | 17.2 | 2.15 | 8.0 | >20 | >20 |
| E000389-202 | DSPC:Chol:DSPE- | citrate | Very fast | 58 | 18.4 | 1.15 | 16.1 | 3.765 | 0.562 |
| | PEG(2000) 55:40:5 | | | | | | | | |
| E000389-203 | DSPC:Chol:DSPE- | sulfate | Very fast | 58.2 | 20.6 | 1.74 | 11.9 | 3.204 | 0.470 |
| | PEG(2000) 55:40:5 | | | | | | | | |
| E000389-204 | DSPC:Chol:DSPE- | sulfate | fast | 60.1 | 18.1 | 1.45 | 12.5 | 3.599 | 0.341 |
| | PEG(2000) 55:40:5 | | | | | | | | |
| E000389-256 | DSPC:Chol:DSPE- | sulfate | ND | 60.3 | 23.6 | 2.28 | 10.3 | 0.365 | 0.054 |
| | PEG(2000) 56:41:3 | | | | | | | | |
| E000389-257 | DSPC:Chol:DSPE- | sulfate | ND | 59.3 | 18.7 | 1.79 | 10.4 | 0.524 | 0.054 |
| | PEG(2000) 56:41:3 | | | | | | | | |
| E000389-252 | DSPC:Chol:DSPE- | sulfate | ND | 98.1 | 23.3 | 3.27 | 7.1 | 0.472 | 0.154 |
| | PEG(5000) 55:40:5 | | | | | | | | |
| E000389-253 | DSPC:Chol:DSPE- | sulfate | ND | 100 | 19.4 | 2.67 | 7.3 | 0.515 | 0.194 |
| | PEG(5000) 55:40:5 | | | | | | | | |
| E000389-254 | DSPC:Chol:DSPE- | citrate | ND | 100.7 | 26 | 3.57 | 7.3 | 0.341 | 0.458 |
| | PEG(5000) 55:40:5 | | | | | | | | |
| E000389-255 | DSPC:Chol:DSPE- | citrate | ND | 101.9 | 19.4 | 2.40 | 8.1 | >20 | 0.129 |
| | PEG(5000) 55:40:5 | | | | | | | | |
| E000389-146 | DSPC:Chol:DSPE- | sulfate | Slow | 99 | 21.8 | 3.32 | 6.6 | >30 | 3.54 |
| | PEG(2000) 55:40:5 | | | | | | | | |
| E000389-149 | DSPC:Chol:DSPE- | citrate | Moderate | 101.3 | 18 | 2.07 | 8.7 | >30 | 11.56 |
| | PEG(2000) 55:40:5 | | | | | | | | |
| E000389-150 | DSPC:Chol:DSPE- | sulfate | Slow | 100.2 | 21.4 | 3.57 | 6.0 | >30 | 0.89 |
| | PEG(2000) 55:40:5 | | | | | | | | |
| E000389-151 | DSPC:Chol:DSPE- | sulfate | Moderate | 102.8 | 17.1 | 2.43 | 7.0 | >30 | >30 |
| | PEG(2000) 55:40:5 | | | | | | | | |
| E000389-166 | DSPC:Chol:DSPE- | sulfate | Moderate | 100.6 | 17.8 | 1.92 | 9.3 | >30 | 3.97 |
| | PEG(2000) 55:40:5 | | | | | | | | |
| E000389-218 | HSPC:Chol:DSPE- | sulfate | ND | 93.4 | 20.80 | 3.95 | 5.3 | 0.310 | 0.358 |
| | PEG(2000)55:40:5 | | | | | | | | |
| E000389-219 | HSPC:Chol:DSPE- | sulfate | Moderate | 95.4 | 16.14 | 2.46 | 6.6 | 0.116 | 0.287 |
| | PEG(2000)55:40:5 | | | | | | | | |
| E000389-220 | HSPC:Chol:DSPE- | citrate | Slow | 91.2 | 21.36 | 4.11 | 5.2 | 0.359 | 0.119 |
| | PEG(2000)55:40:5 | | | | | | | | |
| E000389-221 | HSPC:Chol:DSPE- | citrate | Moderate | 93.9 | 18.87 | 2.69 | 7.0 | 0.424 | 0.151 |
| | PEG(2000)55:40:5 | | | | | | | | |
| E000389-224 | Sphingo:Chol:DSPE- | sulfate | Moderate | 108.1 | 27.20 | 2.99 | 9.1 | 0.395 | 0.105 |
| | PEG(2000)55:40:5 | | | | | | | | |
| E000389-225 | Sphingo:Chol:DSPE- | sulfate | Fast | 107.4 | 18.28 | 2.2 | 8.3 | 0.379 | 0.121 |
| | PEG(2000)55:40:5 | | | | | | | | |
| E000389-226 | Sphingo:Choi:DSPE- | citrate | Moderate | 101.5 | 22.05 | 3.09 | 7.1 | 0.336 | 0.191 |
| | PEG(2000)55:40:5 | | | | | | | | |
| E000389-227 | Sphingo:Chol:DSPE- | citrate | Moderate | 103.3 | 19.60 | 2.26 | 8.7 | 0.406 | 0.285 |
| | PEG(2000)55:40:5 | | | | | | | | |
| E000389-236 | DPPC:Chol:DSPE- | sulfate | ND | 97.8 | 23.4 | 4.07 | 5.7 | 0.296 | 0.269 |
| | PEG(2000)55:40:5 | | | | | | | | |
| E000389-237 | DPPC:Chol:DSPE- | sulfate | Slow | 101.9 | 18.6 | 2.32 | 8.0 | 0.381 | 0.165 |
| | PEG(2000)55:40:5 | | | | | | | | |
| E000389-238 | DPPC:Chol:DSPE- | citrate | Moderate | 93.1 | 21.9 | 3.59 | 6.1 | >20 | 1.486 |
| | PEG(2000)55:40:5 | | | | | | | | |
| E000389-239 | DPPC:Chol:DSPE- | citrate | Moderate | 96.8 | 19.7 | 2.47 | 8.0 | >20 | 0.744 |
| | PEG(2000)55:40:5 | | | | | | | | |
| E000389-340 | DMPC:Chol:DSPE- | sulfate | Moderate | 94.1 | 21.12 | 3.62 | 5.8 | 0.408 | 0.054 |
| | PEG(2000) 55:40:5 | | | | | | | | |
| E000389-341 | DMPC:Chol:DSPE- | sulfate | Moderate | 97.7 | 17.78 | 2.20 | 8.1 | 0.248 | 0.063 |
| | PEG(2000) 55:40:5 | | | | | | | | |
| E000389-342 | DMPC:Chol:DSPE- | citrate | Very fast | 89.3 | 23.59 | 3.43 | 6.9 | 0.342 | 0.208 |
| | PEG(2000) 55:40:5 | | | | | | | | |
| E000389-343 | DMPC:Chol:DSPE- | citrate | Fast | 92.9 | 20.27 | 2.34 | 8.7 | 0.500 | 0.032 |
| | PEG(2000) 55:40:5 | | | | | | | | |
| E000389-351 | POPC:Chol:DSPE- | sulfate | Very fast | 89.7 | 21.62 | 3.61 | 6.0 | 0.117 | 0.061 |
| | PEG(2000) 55:40:5 | | | | | | | | |
| E000389-352 | POPC:Chol:DSPE- | sulfate | Very fast | 91.7 | 16.78 | 2.20 | 7.6 | 0.083 | 0.047 |
| | PEG(2000) 55:40:5 | | | | | | | | |
| E000389-353 | POPC:Chol:DSPE- | citrate | Very fast | 88.6 | 17.11 | 3.74 | 4.6 | 0.234 | 0.191 |
| | PEG(2000) 55:40:5 | | | | | | | | |
| E000389-354 | POPC:Chol:DSPE- | citrate | Very fast | 85.1 | 18.16 | 2.15 | 8.5 | 0.272 | 0.053 |
| | PEG(2000) 55:40:5 | | | | | | | | |
| E000389-355 | DiC20PC:Chol:DSPE- | sulfate | Fast | 94.5 | 21.78 | 3.67 | 5.9 | 3.031 | 0.281 |
| | PEG(2000) 55:40:5 | | | | | | | | |
| E000389-356 | DiC20PC:Chol:DSPE- | sulfate | Moderate | 97.7 | 17.32 | 2.16 | 8.0 | 1.285 | 0.353 |
| | PEG(2000) 55:40:5 | | | | | | | | |
| E000389-357 | DiC20PC:Chol:DSPE- | citrate | Moderate | 91.1 | 24.99 | 3.66 | 6.8 | 2.938 | 0.371 |
| | PEG(2000) 55:40:5 | | | | | | | | |
| E000389-358 | DiC20PC:Chol:DSPE- | citrate | Moderate | 94.4 | 20.36 | 2.26 | 9.0 | >20 | 0.273 |
| | PEG(2000) 55:40:5 | | | | | | | | |
| E000389-359 | DEPC:Chol:DSPE- | sulfate | Very fast | 95.6 | 24.34 | 3.78 | 6.4 | 0.335 | 0.060 |
| | PEG(2000) 55:40:5 | | | | | | | | |
| E000389-360 | DEPC:Chol:DSPE- | sulfate | Very fast | 98.1 | 19.08 | 2.22 | 8.6 | 0.122 | 0.079 |
| | PEG(2000) 55:40:5 | | | | | | | | |
| E000389-361 | DEPC:Chol:DSPE- | citrate | Very fast | 87.4 | 25.58 | 3.88 | 6.6 | 0.300 | 0.095 |
| | PEG(2000) 55:40:5 | | | | | | | | |
| E000389-362 | DEPC:Chol:DSPE- | citrate | Very fast | 89.1 | 20.41 | 2.20 | 9.3 | 0.180 | 0.149 |
| | PEG(2000) 55:40:5 | | | | | | | | |

Although the foregoing has been described in some detail by way of illustration and example for purposes of clarity and understanding, one of skill in the art will appreciate that certain changes and modifications can be practiced within the scope of the appended claims. In addition, each reference provided herein is incorporated by reference in its entirety to the same extent as if each reference was individually incorporated by reference.

What is claimed is:

1. A liposomal composition for the treatment of cancer in a patient in need thereof comprising:
a) a liposome comprising:
i) about 50 mol % to about 70 mol % of a phosphatidylcholine lipid or mixture of phosphatidylcholine lipids;
ii) about 25 mol % to about 45 mol % of cholesterol;
iii) about 2 mol % to about 8 mol % of a PEG-lipid; and
iv) elinafide; and
b) a pharmaceutically acceptable excipient.

2. The liposomal composition of claim 1, wherein the phosphatidylcholine lipid is selected from the group consisting of: palmitoyloleoylphosphatidylcholine (POPC), distearoylphosphatidylcholine (DSPC), dimyristoylphosphatidylcholine (DMPC), dipalmitoylphosphatidylcholine (DPPC), hydrogenated soy phosphatidylcholine (HSPC), 1,2-diarachidoyl-sn-glycero-3-phosphocholine, and mixtures thereof.

3. The liposomal composition of claim 2, wherein the phosphatidylcholine lipid is DSPC.

4. The liposomal composition of claim 1, wherein the PEG-lipid is selected from the group consisting of distearoyl-phosphatidylethanolamine-N-[methoxy(polyethene glycol)-2000](DSPE-PEG-2000) and distearoyl-phosphatidylethanolamine-N-[methoxy(polyethene glycol)-5000] (DSPE-PEG-5000).

5. The liposomal composition of claim 1, wherein the liposome comprises about 55% of DSPC; about 40% cholesterol; and about 5% DSPE-PEG-2000.

6. The liposomal composition of claim 1, wherein the ratio of the total lipid weight to the elinafide is about 1:0.1 to 1:1.

7. The liposomal composition of claim 1, wherein the liposome has a volume mean particle size of about 125 nm or less.

8. The liposomal composition of claim 7, wherein the liposome has a volume mean particle size of about 50 nm about 100 nm.

9. The liposomal composition of claim 1, wherein less than 10% of the elinafide is released from the liposome within 20 hours.

10. The liposomal composition of claim 1, wherein the composition has an IC₅₀ of about 10.0 μM or less after 2 hours in HT29 cells.

11. The liposomal composition of claim 1, wherein the composition has an IC₅₀ of about 1.0 μM or less after 24 hours in HT29 cells.

12. The liposomal composition of claim 1, wherein the composition has a plasma AUC greater than free elinafide or a clearance rate (Cl) less than free elinafide.

13. The liposomal composition of claim 1, wherein composition inhibited tumor growth greater than free elinafide.

14. The liposomal composition of claim 1, wherein the composition delayed tumor growth greater than free elinafide.

15. The liposomal composition of claim 1, wherein the composition does not induce or has reduced muscle myopathy compared to free elinafide.

16. A method of treating cancer in a patient in need thereof, comprising the steps of:
(a) administering to the patient in need thereof a liposomal composition comprising
i) a liposome comprising:
1) about 50 mol % to about 70 mol % of a phosphatidylcholine lipid or mixture of phosphatidylcholine lipids;
2) about 25 mol % to about 45 mol % of cholesterol;
3) about 2 mol % to about 8 mol % of a PEG-lipid; and
4) elinafide; and
ii) a pharmaceutically acceptable excipient;
wherein the administration of the liposomal composition has a reduced occurrence of muscle myopathy in the patient.

17. The method of treating cancer of claim 16, wherein an initial dose of the liposomal composition is from about 0.001 mg/kg to about 1000 mg/kg daily.

18. The method of treating cancer of claim 17, wherein the initial dose of the liposomal composition is from about 1 g/kg to about 100 mg/kg daily.

19. The method of treating cancer of claim 16, wherein a daily dose of the liposomal composition is 3, 6, 12, 24, 48, 80, 120, 160, 190, 225, 270, or 320 mg/M².
